(19) United States
(12) Patent Application Publication
(10) Pub. No.: US 2024/0336713 A1
(43) Pub. Date: Oct. 10, 2024

(54) CO-OLIGOMERIZATION METHOD EMBODIMENTS FOR PRODUCING JET-RANGE OLEFINS SUITABLE FOR MAKING JET FUEL

(71) Applicant: Battelle Memorial Institute, Richland, WA (US)

(72) Inventors: Karthikeyan Kallupalayam Ramasamy, West Richland, WA (US); Mond F. Guo, Richland, WA (US); Udishnu Sanyal, Richland, WA (US); Robert Dagle, Richland, WA (US); Johnny Saavedra Lopez, Richland, WA (US); Vanessa Dagle, Richland, WA (US)

(73) Assignee: Battelle Memorial Institute, Richland, WA (US)

(21) Appl. No.: 18/296,564

(22) Filed: Apr. 6, 2023

(51) Int. Cl. C08F 10/02; B01J 21/08; B01J 29/40; C08F 4/02; C08F 10/06

(52) U.S. Cl. CPC C08F 10/02; B01J 21/08; B01J 29/40; C08F 4/025; C08F 10/06; C08F 2410/04

(57) ABSTRACT

Disclosed herein are embodiments of a method for making jet-range olefins using co-oligomerization of a mixed olefin composition as a feedstock. In some embodiments, C₂ and C₃₊ olefins can be reacted in the presence of a hybrid catalyst system to produce a jet-range olefin product suitable for use in making jet fuel. In some additional embodiments, C₂ and C₄ olefins can be reacted in the presence of a single catalyst component to produce a jet-range and/or diesel-range olefin product suitable for use in making jet fuel. Combinations comprising catalysts and feedstocks disclosed herein also are described, along with system embodiments for conducting the co-oligomerization method embodiments.

### ACKNOWLEDGMENT OF GOVERNMENT SUPPORT

This invention was made with government support under Contract DE-AC05-76RL01830 awarded by the U.S. Department of Energy. The government has certain rights in the invention.

### FIELD

Method and system embodiments for converting feedstocks comprising mixture of light olefins into jet-range olefins via co-oligomerization using hybrid catalyst system embodiments are disclosed.

### BACKGROUND

Despite recent development of effective heterogeneous approaches to ethylene conversion, currently there exists a significant technological gap concerning the co-oligomerization of ethylene with C₃₊ olefins and a catalytic approach that can effectively co-oligomerize mixed olefins to form jet-range olefins in high yield. A need exists in the art for a method that can be used to arrive at products that can be used to provide jet fuel hydrocarbons with high yield and selectivity.

### SUMMARY

Disclosed herein are embodiments of a method, comprising: contacting, in a single reactor, a feedstock comprising a mixture of C₂ and C₃₊ olefins with a hybrid catalyst system comprising a transition-metal based catalyst and an acid catalyst, and heating the single reactor at a temperature, such as at a temperature ranging from 250° C. to 500° C., to convert the feedstock into an oligomerized product composition comprising jet-range olefins via co-oligomerization.

Also disclosed herein are embodiments of a reactor for performing the method as described herein, where the reactor comprising a hybrid catalyst system comprising a transition-metal based catalyst and an acid catalyst, wherein the transition-metal based catalyst and the acid catalyst are provided on separate catalyst beds or on the same catalyst bed and the hybrid catalyst system is provided in an amount sufficient to convert C₂ and C₃₊ olefins into jet-range olefins via co-oligomerization.

Also disclosed herein are embodiments of a combination comprising a feedstock comprising a mixture of C₂ and C₃₊ olefins, and a hybrid catalyst system comprising a transition-metal based catalyst and an acid catalyst.

Also disclosed herein are embodiments of a method, comprising contacting, in a single reactor, a feedstock comprising a mixture of C₂ and C₄ olefins with a single catalyst component comprising a transition-metal based catalyst and an acid catalyst, and heating the single reactor at a temperature ranging from 150° C. to 300° C. to convert the feedstock into an oligomerized product composition comprising jet-range and/or diesel-range olefins via co-oligomerization.

The foregoing and other objects, features, and advantages of the present disclosure will become more apparent from the following detailed description, which proceeds with reference to the accompanying figures.

### BRIEF DESCRIPTION OF THE DRAWINGS

FIGS. 1A-1D provide schematic flow diagrams showing different potential pathways to C₂₊ mixed olefin intermediates that can be oligomerized to produce sustainable aviation fuel (also referred to herein as “SAF”) using method and system embodiments of the present disclosure, wherein: FIG. 1A shows MTO-based pathway through a syngas and methanol intermediate; FIG. 1B shows dehydration of small-oxygenates produced through fermentation; FIG. 1C shows cracking of naphtha streams from fractionation of bio-fuels produced from processes such as catalytic fast pyrolysis; and FIG. 1D shows hydrotreating waste and biomass sources.

FIG. 2A-2C provide schematic diagrams describing aspects of oligomerization mechanisms using different feedstocks, such as (i) ethylene, which is oligomerized through the transition metal-catalyzed Cossee-Arlman mechanism to produce linear products (FIG. 2A); (ii) C₃₊ olefins, which undergo acid-catalyzed oligomerization to yield branched products (FIG. 2B); and (iii) a process according to the present disclosure whereby a mixed olefin feed can be used to arrive at desired oligomerized products (FIG. 2C).

FIGS. 3A-3D are schematic diagrams showing possible process pathways using co-oligomerization of mixed olefin feeds (e.g., C₂₊ olefin feeds), wherein FIG. 3A shows a single-pass C₂₊ olefin oligomerization to jet-range olefins using a single catalyst bed comprising a hybrid catalyst system without recycling; FIG. 3B shows a single-pass C₂₊ olefin oligomerization to jet-range olefins using a dual-bed setup comprising a hybrid catalyst system without recycling; FIG. 3C shows hybrid C₂₊ olefin oligomerization to jet-range olefins in a single catalyst bed comprising a hybrid catalyst system with separation and recycling of olefins having carbon numbers lower than 8; and FIG. 3D shows hybrid C₂₊ olefin oligomerization to jet-range olefins in a dual-bed setup comprising a hybrid catalyst system with separation and recycling of olefins having carbon numbers lower than 8.

FIG. 4 is a piping and instrumentation diagram (P&ID) showing components and connections of a representative system used for oligomerizing mixed olefin streams according to the present disclosure.

FIG. 5 is a graph showing results from catalyst reactivity testing of a nickel ion-exchanged amorphous silica alumina, wherein the catalyst was pretreated under flowing N₂ for 3 hours at 300° C.

FIGS. 6A and 6B are graphs showing results from co-oligomerizing an ethylene/propylene mixture over a Ni/SiO₂—Al₂O₃ catalyst at 200° C. and 275° C., wherein FIG. 6A is a graph of conversion (%) as a function of time on stream (TOS); and FIG. 6B is a graph of product selectivity (%) as a function of carbon number.

FIGS. 7A and 7B are graphs showing results from co-oligomerizing an ethylene/propylene mixture using a hybrid catalyst system comprising Ni/SiO₂—Al₂O₃ and ZSM-5 zeolite, wherein the hybrid catalyst system is embedded on a single catalyst bed at 275° C., wherein FIG. 7A is a graph of conversion (%) as a function of time on stream (TOS); and FIG. 7B is a graph of product selectivity (%) as a function of carbon number.

FIG. 8 is a graph showing results from co-oligomerizing an ethylene/propylene mixture over a hybrid catalyst system comprising Ni/SiO₂—Al₂O₃ and ZSM-5 catalysts with varying Si/Al ratio, wherein the bar graph shows the overall C₂/C₃ conversion and circle shows the total product selectivity corresponding to C₈-C₁₆, which is a range of olefins suitable for making jet fuel.

FIG. 9 is a graph showing results from co-oligomerizing an ethylene/propylene mixture over a hybrid catalyst system comprising Ni/SiO₂—Al₂O₃ and MFI catalyst at 275° C. and 100 psig pressure, with varying ratio of ethylene and propylene, wherein the bar graph shows the product selectivity (%) as a function of varying propylene to ethylene ratio.

FIG. 10 shows results obtained using gas chromatograph/mass spectrometry (GCMS) to analyze reaction content during co-oligomerization of an ethylene/propylene mixture over a hybrid catalyst system comprising Ni/SiO₂—Al₂O₃ and ZSM-5 catalyst at 275° C. and 100 psig pressure, with a varying ratio of ethylene and propylene, which demonstrates very similar product distribution irrespective of their feed composition.

FIG. 11 is a graph showing product selectivity results from co-oligomerizing different feedstock compositions at different temperatures over a hybrid catalyst system comprising Ni/SiO₂—Al₂O₃ and ZSM-5 catalysts at 100 psig pressure and 0.8 h⁻¹ WHSV.

FIG. 12 is a graph showing product selectivity results from co-oligomerizing feedstock compositions with different C₂:C₃:C₄:C₅ ratios at 250° C. and 100 psig pressure over a hybrid catalyst system comprising Ni/SiO₂—Al₂O₃ and ZSM-5 catalysts with 0.8 h⁻¹ WHSV.

FIG. 13 is a graph showing a simulated distillation profile for the hydrocarbon liquid product resulting from an oligomerization of an olefin mixture comprising butene and ethene at a 4/1 molar ratio in the presence of N₂ (63 vol. % of butene) over BEA and 5 wt. % Ni/BEA using operating parameters of 230° C. and 200 psig; the reaction conditions for this example include 2 g of catalyst, 0.11 mL/min butene feed, WHSV=2.0 h⁻¹ (g_{butene}/g_{cat}/h).

FIG. 14 is a graph showing a GC-MS analysis result for the liquid products produced from BEA zeolite with Ni promotion (5 wt. % Ni). This graph shows carbon number and hydrocarbon classification of the liquid products.

### DETAILED DESCRIPTION

### I. Overview of Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Also, the following description is exemplary in nature and is not intended to limit the scope, applicability, or configuration of the present disclosure. Various changes to the described embodiment may be made in the function and arrangement of the elements described herein without departing from the scope of the preset disclosure. Further, descriptions and disclosures provided in association with one particular embodiment are not limited to that embodiment and may be applied to any embodiment disclosed. Further, the terms “coupled” and “associated” generally mean fluidly, electrically, and/or physically (e.g., mechanically or chemically) coupled or linked and does not exclude the presence of intermediate elements between the coupled or associated items absent specific contrary language.

Although the operations of exemplary embodiments of the disclosed method and/or system embodiments may be described in a particular, sequential order for convenient presentation, it should be understood that disclosed embodiments can encompass an order of operations other than the particular, sequential order disclosed, unless the context dictates otherwise. For example, operations described sequentially may in some cases be rearranged or performed concurrently. Further, descriptions and disclosures provided in association with one particular embodiment are not limited to that embodiment and may be applied to any disclosed embodiment.

To facilitate review of the various embodiments of the disclosure, the following explanations of specific terms are provided.

Co-oligomerization: A chemical process that oligomerizes a feedstock comprising a mixture of (i) C₂ olefins and (ii) C₃₊ olefins. In particular embodiments, the co-oligomerization takes place in a single reactor using a hybrid catalyst system that can be provided on a single catalyst bed or a dual catalyst bed.

Diesel-Range Olefins: A component of an oligomerized product mixture according to the present disclosure that comprises one or more olefins having at least 11 carbon atoms and typically up to 23 carbon atoms.

Hybrid catalyst system: A catalyst system having at least two different catalysts. In some embodiments, the hybrid catalyst system can comprise a physical mixture of the at least two different catalysts. In other embodiments, the hybrid catalyst system can comprise the at least two different catalysts as physically separate components. The hybrid catalyst system typically comprises a first catalyst that is a transition metal-based catalyst and a second catalyst that is an acid catalyst.

Jet fuel: A hydrocarbon or hydrocarbon mixture that distills at a temperature ranging from 120° C. to 300° C. In some embodiments, these types of hydrocarbons can include those with a carbon number between C₈ and C₁₆; however, actual range limits for commercial use can depend on other required fuel properties.

Jet-Range Olefins: A component of an oligomerized product mixture according to the present disclosure that comprises one or more olefins having at least eight carbon atoms and typically up to 16 carbon atoms.

Mobil-type five (MFI) zeolites: Zeolites with structure type “MFI,” which are three dimensional microporous aluminosilicates with pore openings of 5.5 Å.

Non-jet-range product: A hydrocarbon or hydrocarbon mixture comprising at least some olefins that can be generated by the co-oligomerization process according to the present disclosure, along with the jet-range olefins. In some embodiments, non-jet-range products can include olefins having carbon numbers less than 8.

Olefin/Alkene: An unsaturated hydrocarbon containing at least one double bond positioned along the length of the hydrocarbon chain. The hydrocarbon chain may be straight (i.e., acyclic, linear, or normal), cyclic, or branched (e.g., containing one or more hydrocarbon sidechains). As used herein, “C₂ olefin” refers to an olefin having two carbon atoms (also referred to herein as “ethylene”); “C₃₊ olefins” refers to a mixture of olefins, wherein each olefin has at least three carbon atoms.

Oligomerization: A chemical process that converts lower olefins to higher carbon-number olefins that can be used as fuels, such as aviation fuel, via chemical reaction(s).

Single-pass conversion: A yield of product obtained after one pass through a system/oligomerization method according to the present disclosure. The yield is calculated as: difference between the C₂ and C₃ olefins in reactor feed and the C₂ and C₃ olefins in reactor outlet, divided by the C₂ and C₃ olefins in reactor feed.

Syngas or Synthesis Gas: A fuel gas mixture typically made up of hydrogen, carbon monoxide, and, optionally, carbon dioxide.

Transition Metal-Based Catalyst: A catalyst comprising a metal component and a support component. The support component of any transition metal-based catalyst may be similar in chemical make-up as a solid acid catalyst as described herein; however, the support component of the transition metal-based catalyst is a separate component than the acid catalyst component of any hybrid catalyst system.

ZSM-5 or ZSM-5 zeolites: ZSM-5 (which stands for Zeolite Socony Mobil-five) is an aluminosilicate zeolite containing a three-dimensional channel system. The chemical formula of ZSM-5 is NaₙAlₙSi₉₆₋ₙO₁₉₂·16H₂O (wherein 0<n<27). The typical pore size is 5.5 Å.

### II. Introduction

Disclosed herein are embodiments of a method for converting a feedstock comprising a mixture of C₂ and C₃₊ olefins into jet-range olefins via co-oligomerization using a hybrid catalyst system, as well as embodiments of a system for carrying out such method embodiments. In particular embodiments, the method uses a single reactor to convert C₂ and C₃₊ olefins to jet-range products.

Also disclosed here are embodiments of a method for converting a feedstock comprising a mixture of C₂ and C₄ olefins into jet-range and/or diesel-range olefins via co-oligomerization using a single catalyst component, as well as embodiments of a system for carrying out such method embodiments. In particular embodiments, the method is able to produce jet-range olefins in a selective single-step oligomerization.

To meet the immediate need for decarbonization of the aviation industry, leveraging existing commercial processes and feedstocks will be the most efficient path toward producing SAF in the near-term future. Syngas is one of the most attractive feed sources as it can be derived from a broad range of renewable and waste feedstocks via gasification, while benefiting from existing infrastructure throughout the petrochemical industry. This general applicability makes syngas an effective vehicle for accessing a wide range of renewable feedstocks where heterogeneity precludes the use of conventional conversion pathways. These include ecologically disadvantaged feedstocks such as municipal solid wastes (MSW) and residual biomass that can offer significant carbon reductions.

Of the existing industrial processes for transforming syngas to synthetic fuels, none produce aviation fuel efficiently. Several industrial syngas-based technologies have been developed to produce synthetic fuels, based on either direct conversion through the Fischer-Tropsch (FT) chemistry or indirect conversion via the synthesis of a methanol intermediate. Commercial FT processes, which include the Shell Middle Distillate Synthesis (SMDS) and Conversion to Distillates (COD) developed by PetroSA, typically involve high capital costs due to complex reactor design and require additional unit operations to upgrade wax byproducts. Methanol-based technologies include Mobil's Methanol-to-Gasoline (MTG) and Mobil-Olefins-to-Gasoline- and-Distillate (MOGD) processes. The MTG process is limited to the production of ≤C₁₀ hydrocarbons and effluent streams typically contain substantial amounts of aromatics. Modification of MTG operating conditions and unit operations results in the MOGD process, which allows for the production of diesel-range hydrocarbons at higher costs but still generates a significant fraction of unwanted gasoline-range aromatics. Overall, these commercialized processes offer little market penetration, and none of them provide a high selectivity to jet-range products despite their operational complexity.

Table 1 provides a comparison of yields obtained using syngas-based conversion technology currently existing in the art to generate SAF and an exemplary yield obtained using a method embodiment according to the present disclosure.

TABLE 1
Comparison between various syngas-to-fuel technologies and their jet-range yields

| | Preliminary Process | | | | | |
|---|---|---|---|---|---|---|
| | Yields (%) | | Process Yield (%) | | | Total Jet- |
| | Syngas-to- | | | Jet- | | Range Yield |
| Technology | MeOH | MTO | Gasoline | Range | Diesel | (%) |
| FT | — | — | 40 | 52 | 7 | 52 |
| COD | — | — | 26 | — | 51 | — |
| SMDS | — | — | 25 | 50 | 25 | 50 |
| TIGAS | — | — | 80 | — | — | — |
| MTG | 99 | — | 81 | — | — | — |
| MOGD | 99 | — | 18 | 24 | 55 | 24 |
| Representative method | 99 | 92 | 0 | 80 | 20 | 73 |
| embodiment | | | | | | |

FT: Fischer-Tropsch,
COD: Conversion of Distillates,
SMDS: Shell Middle Distillate Synthesis,
TIGAS: Topsøe Integrated Gasoline Synthesis,
MTG: Methanol-to-Gasoline,
MOGD: Mobil-Olefins-to-Gasoline-and-Distillate

Methanol synthesis followed by methanol-to-olefin (MTO) processing offers an already established and active commercialized pathway to produce mixed light olefins, primarily ethylene and propylene. This mixture of light olefins can potentially be directly oligomerized to jet-range products in a single reaction step; however, the difference in reactivity and corresponding oligomerization pathways between C₂ and C₃₊ olefins make this challenging, particularly in view of the well-known difficulty of converting ethylene, which exhibits a relatively high stability compared to other olefins.

Generating mixed olefin mixtures with average carbon numbers of 3 to 4, possible from a number of different pathways such as MTO, opens the possibility for a significant reduction in process complexity such as using single-step oligomerization approach to generating jet-range products that can be converted to jet fuel. Such an approach will require a correspondingly high conversion of the ethylene fraction in the same reaction step.

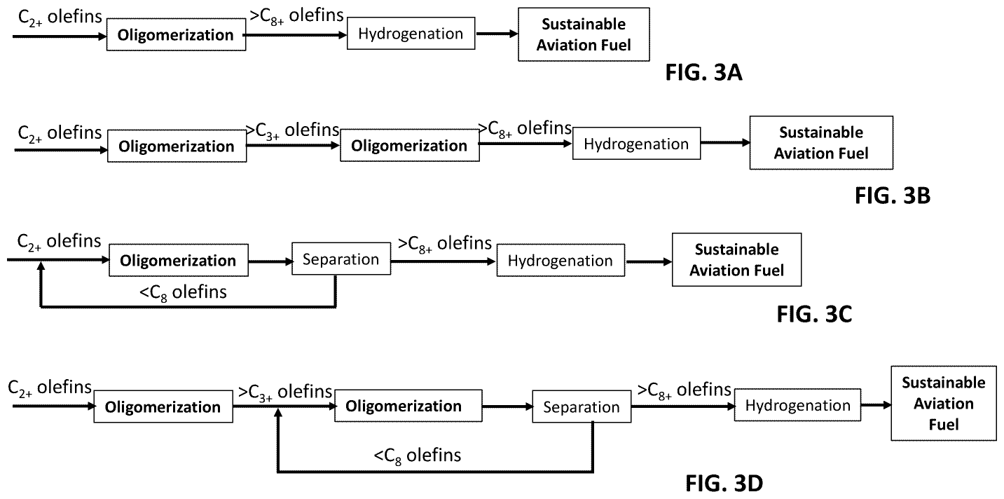

A significant technological gap regarding the co-oligomerization of ethylene with C₃₊ olefins exists in the art. The present disclosure, however, describes method embodiments that facilitate making jet-range products that can be converted to jet fuel, wherein the method uses feedstocks comprising C₂ and C₃₊ olefin mixtures. In particular embodiments, a hybrid catalyst system is used and maintains the overall improvement in process intensity while still allowing for independent optimization of the Cossee-Arlman and acid-catalyzed reactions. FIGS. 3A-3D summarize exemplary method embodiments contemplated by the present disclosure. These embodiment variations offer flexibility to adapt feed variability and utilize a core C₂₊ co-oligomerization technology. As the method embodiments of the present disclosure are more efficient than conventional methods, they can be used to lower the need for larger recycle loops and make single-pass processes more viable.

### III. Method Embodiments

Embodiments of a method for converting feedstocks into an oligomerized product composition via co-oligomerization are disclosed. The oligomerized product composition comprises jet-range olefins that can be converted to jet fuel. In some embodiments, the method includes contacting a feedstock with a novel hybrid catalyst system to convert the feedstock into the oligomerized product composition via co-oligomerization in a single reactor comprising (i) a single catalyst bed system comprising a hybrid catalyst system (e.g., wherein components of the hybrid catalyst system are mixed within/on a single catalyst bed); or (ii) a dual catalyst bed system comprising the hybrid catalyst system (e.g., wherein components of the hybrid catalyst system are placed within/on separate catalyst beds in sequential order). In some other embodiments, the method includes contacting a feedstock with a single catalyst component to convert the feedstock into the oligomerized product composition via co-oligomerization in a single reactor comprising a single catalyst bed system comprising the single catalyst component.

Other aspects of the disclosed method and system embodiments are described herein.

### A. Feedstock and Feedstock Pathways/Sources

In some embodiments, the feedstock comprises mixed olefins. In certain embodiments, the mixed olefins include a mixture of C₂ and C₃₊ olefins. C₃₊ olefins can comprise C₃ olefins, C₄ olefins, C₅ olefins, C₆ olefins, Ce olefins, and any combinations thereof.

In some embodiments, the mixture of C₂ and C₃₊ olefins includes C₂ to C₅ olefins. In one embodiment, the mixture of C₂ and C₃₊ olefins comprises C₂ and C₄ olefins. In another embodiment, the mixture of C₂ and C₃₊ olefins consists essentially of C₂ and C₄ olefins. In yet another embodiment, the mixture of C₂ and C₃₊ olefins consists of C₂ and C₄ olefins.

In some embodiments, the C₃₊ olefins comprise olefins with carbon numbers of 3 or higher. In certain embodiments, the C₃₊ olefins comprise olefins with carbon numbers ranging from 3 to 7. In one embodiment, the C₃₊ olefins comprise a mixture of at least C₃, C₄, and C₅ olefins. In another embodiment, the C₃₊ olefins comprise a majority of C₄ olefins. In a particular embodiment, the C₃₊ olefins consist essentially of C₄ olefins.

In some embodiments, the mixed olefins comprise C₂ and C₄ olefins in mole ratio of from 1:9 to 1:1. In some embodiments, the feedstock comprises a mixture of C₂ and C₄ olefins wherein the C₄ olefins are present in an amount ranging from 5 vol % to 70 vol %. In certain embodiments, the feedstock comprises a mixture of C₂ and C₄ olefins wherein the C₄ olefins are present in an amount ranging from 60 vol % to 65 vol %. In one particular embodiment, the feedstock comprises a mixture of C₂ and C₄ olefins wherein the C₄ olefins are present in an amount of 63 vol %.

In particular embodiments, the feedstock comprising a mixture of C₂ and C₃₊ olefins is derived from a carbonaceous feed source. In one embodiment, the carbonaceous feed source includes coal. In some other embodiments, the carbonaceous feed source includes a heterogeneous carbonaceous feed source. Advantageously, in certain embodiments, the heterogeneous carbonaceous feed source includes ecologically disadvantaged sources such as municipal solid wastes (MSW) and residual biomass that can offer significant carbon reductions. In other embodiments, the heterogeneous carbonaceous feed source includes industrial off-gas and biogas.

In any of the foregoing or following embodiments, the feedstock comprising a mixture of C₂ and C₃₊ olefins is derived from a feed source via a feedstock pathway.

In one embodiment, the feedstock comprising a mixture of C₂ and C₃₊ olefins is derived from a feed source comprising biomass or waste (e.g., municipal solid waste, industrial off-gas, biogas, and the like) via dehydration of small-oxygenates produced through fermentation. In another embodiment, the feedstock comprising a mixture of C₂ and C₃₊ olefins is derived from a feed source comprising biomass via cracking naphtha streams from fractionation of biofuels produced from processes such as catalytic fast pyrolysis. In yet another embodiment, the feedstock comprising a mixture of C₂ and C₃₊ olefins is derived from a feed source comprising waste via hydrotreating the waste.

In some embodiments, the feedstock comprises mixed olefins with a C₃₊:C₂ molar ratio ranging from 9:1 to 1:9, such as from 8:1 to 1:8, or from 7:1 to 1:7, or from 6:1 to 1:6, or from 5:1 to 1:5, or from 4:1 to 1:4, or from 3:1 to 1:3, or from 2:1 to 1:2. In certain embodiments, the feedstock comprises mixed olefins with a C₃₊:C₂ molar ratio ranging from 3:1 to 1:1. In some other embodiments, the feedstock comprises mixed olefins with a C₃₊:C₂ molar ratio ranging from 2:1 to 1:2. In one embodiment, the feedstock comprises mixed olefins with a C₃₊:C₂ molar ratio of 2:1. In another embodiment, the feedstock comprises mixed olefins with a C₃₊:C₂ molar ratio of 3:1. In yet another embodiment, the feedstock comprises mixed olefins with a C₃₊:C₂ molar ratio of 5:1.

In some other embodiments, the feedstock comprises mixed olefins with a C₃₊:C₂ molar ratio ranging from 5:1 to 1:5, wherein the C₃₊ olefins comprises C₃, C₄ and C₅ olefins. In certain embodiments, the feedstock comprises mixed olefins with a C₃₊:C₂ molar ratio ranging from 3:1 to 1:3. In some other embodiments, the feedstock comprises mixed olefins with a C₃₊:C₂ molar ratio ranging from 2:1 to 1:2. In one embodiment, the feedstock comprises mixed olefins with a C₃₊:C₂ molar ratio of 2:1. In another embodiment, the feedstock comprises mixed olefins with a C₃₊:C₂ molar ratio of 3:1. In yet another embodiment, the feedstock comprises mixed olefins with a C₃₊:C₂ molar ratio of 5:1.

In some other embodiments, the feedstock comprises mixed olefins with a C₃₊:C₂ (e.g., C₃:C₂, C₃₊:C₂, or C₄:C₂) molar ratio ranging from 4:1 to 1:4. In certain embodiments, the feedstock comprises mixed olefins with a C₃₊:C₂ (e.g., C₃:C₂, C₃₊:C₂, or C₄:C₂) molar ratio ranging from 3:1 to 1:1. In some other embodiments, the feedstock comprises mixed olefins with a C₃₊:C₂ (e.g., C₃:C₂, C₃₊:C₂, or C₄:C₂) molar ratio ranging from 2:1 to 1:2. In one embodiment, the feedstock comprises mixed olefins with a C₃₊:C₂ (e.g., C₃:C₂, C₃₊:C₂, or C₄:C₂) molar ratio of 2:1.

In some embodiments, the feedstock comprises mixed olefins with a C₂:C₃:C₄:C₅ molar ratio of 2:2:1:1. In some other embodiments, the feedstock comprises mixed olefins with a C₂:C₃:C₄:C₅ molar ratio of 1:1:1:1. In some other embodiments, the feedstock comprises mixed olefins with a C₂:C₃:C₄:C₅ molar ratio of 1:1:2:2.

In particular embodiments, the feedstock comprising a mixture of C₂ and C₃₊ olefins is derived from a feed source comprising syngas via a methanol-to-olefin based (MTO) pathway comprising methanol synthesis followed by an MTO process.

In some embodiments, a carbonaceous feed source is gasified into syngas. In further embodiments, the syngas is reacted over a catalyst at a reaction temperature under a reaction pressure to produce methanol. The catalyst may comprise a Cu catalyst, ZnO catalyst, or Al₂O₃ catalyst. In some embodiments, the reaction temperature ranges from 200° C. to 300° C., such as 230° C. to 275° C. or 250° C. to 270° C. In some embodiments, the reaction pressure ranges from 40 bar to 70 bar, such as 50 bar to 60 bar, or 55 bar to 60 bar. In some embodiments, the methanol selectivity is greater than 90%, such as greater than 95%, or greater than 99%.

In some embodiments, the methanol to olefin process comprises react methanol with a catalyst at a reaction temperature under a reaction pressure to produce olefins. In certain embodiments, the catalyst is a SAPO-34 catalyst. In some embodiments, the reaction temperature ranges from 400° C. to 500° C., such as from 430° C. to 470° C., or 450° C. to 470° C. In some embodiments, the reaction pressure ranges from 40 bar to 70 bar, such as 50 bar to 60 bar, or 55 bar to 60 bar. In some embodiments, the olefins comprise small olefins. In certain embodiments, the olefins comprise ethylene and propylene. In one embodiment, the selectivity of ethylene and propylene to total olefins is greater than 80%, such as greater than 90%, or greater than 92%.

### B. Co-oligomerization of C₂ and C₃₊ olefins

Embodiments of a method for converting feedstocks into an oligomerized product composition via co-oligomerization are disclosed. The oligomerized product composition is suitable for conversion to jet fuel.

In some embodiments, the method comprises contacting, in a single reactor, a feedstock with a single catalyst component and heating the single reactor to convert the feedstock into an oligomerized product composition comprising jet-range olefins (and, in some embodiments, diesel-range olefins) via co-oligomerization.

In some other embodiments, the method comprises contacting, in a single reactor, a feedstock with a hybrid catalyst system and heating the single reactor to convert the feedstock into an oligomerized product composition comprising jet-range olefins via co-oligomerization. FIG. 3A shows one exemplary embodiment of the method. FIG. 3A shows full conversion in the oligomerization of ethylene as well as higher C₃₊ olefins in a single-pass process using a hybrid catalyst system in a single reactor comprising a single- or dual-bed configuration, promoting both Cossee-Arlman and acid-based oligomerization reactions in the single reactor.

In some embodiments, the oligomerized product composition further comprises non-jet-range products, such as non-jet-range olefins. In certain embodiments, the non-jet-range products comprise olefins having carbon numbers below C₈. In certain embodiments, non-jet-range products include C₂-C₇ olefins. In other embodiments, the non-jet-range products include C₃₊ olefins. In one embodiment, the non-jet-range products include C₃-C₇ olefins.

In some embodiments, the single reactor is operated or heated at a temperature ranging from 100° C. to 500° C., such as 100° C. to 450° C., 150° C. to 400° C., 150° C. to 300° C., 150° C. to 500° C., 250° C. to 500° C., 200° C. to 350° C., and 250° C. to 300° C. In certain embodiments, the single reactor is operated at a temperature ranging from 200° C. to 300° C. or 250° C. In yet other embodiments, the single reactor is operated at a temperature ranging from 250° C. to 500° C., such as 250° C. to 450° C., 250° C. to 400° C., or 250° C. to 350° C.

In some embodiments, the olefins are oligomerized under a pressure ranging from 50 psig to 500 psig, such as 50 psig to 450 psig, or 55 psig to 400 psig, or 60 psig to 400 psig, or 70 psig to 350 psig, or 75 psig to 350 psig, or 75 psig to 300 psig, or 75 psig to 200 psig, or 75 psig to 150 psig, or 75 psig to 125 psig. In some embodiments, the pressure can range from 200 psig to 500 psig. In certain embodiments, the olefins are oligomerized under a pressure ranging from 75 to 500 psig, such as from 75 to 300 psig, or 75 psig to 200 psig. In one embodiment, the pressure is 100 psig. In another embodiment, the pressure is 200 psig.

In some embodiments, contacting the feedstock with the hybrid catalyst system comprises contacting the feedstock with a first catalyst bed comprising the transition-metal based catalyst to produce a first product mixture and then contacting the first product mixture with a second catalyst bed comprising the acid catalyst to convert the first product mixture into the oligomerized product composition. In such embodiments, the first catalyst bed and second catalyst bed are contained in the single reactor. FIG. 3B shows one exemplary embodiment of the methods. FIG. 3B shows a second bed added to dimerize the C₃₊ olefins produced in the first reaction step.

In further embodiments, the method further comprises separating the jet-range olefins from any non-jet-range products; and/or contacting the non-jet-range products with the hybrid catalyst system to produce jet-range olefins. FIG. 3D shows one exemplary embodiment of such a method. FIG. 3D shows that olefins having carbon numbers lower than 8 can be recycled directly to the second catalyst bed to improve the process efficiency, to provide even greater control over the resulting jet fuel properties, and higher SAF yields.

In other embodiments, the method comprises contacting (or exposing), in a single reactor, a feedstock comprising a mixture of C₂ and C₃₊ olefins with (or to) the hybrid catalyst system and heating the single reactor to convert the feedstock into an oligomerized product composition comprising jet-range olefins and non-jet-range olefins via co-oligomerization, separating the jet-range olefins from the non-jet-range products, and recycling the non-jet-range products for further oligomerization. In certain embodiments, recycling the non-jet-range products comprises contacting the non-jet-range products with the hybrid catalyst system to produce jet-range olefins. FIG. 3C shows one exemplary embodiment of such a method. FIG. 3C shows olefins having carbon numbers lower than 8 can be recycled to the single catalyst bed to improve the process efficiency, to provide even greater control over the resulting jet fuel properties and provide higher SAF yields.

In some embodiments, the hybrid catalyst system comprises a mixed bed configuration wherein both components of the hybrid catalyst system are contained on a single catalyst bed. In some other embodiments, the hybrid catalyst system comprises a staged bed configuration wherein the two components of the hybrid catalyst system are contained on separate catalyst beds. In some embodiments, the hybrid catalyst system is configured to comprise a catalyst bed comprising Ni/SiO₂—Al₂O₃ catalyst and a zeolite catalyst. In such embodiments, the Ni/SiO₂—Al₂O₃ catalyst is loaded on a catalyst bed positioned near the top of the reactor, and the zeolite catalyst is loaded on a catalyst bed positioned near the bottom of the reactor.

In some embodiments, the disclosed method produces a product stream comprising the oligomerized product composition. In certain embodiments, the oligomerized product composition comprises n- and/or iso-alkenes. In one embodiment, the oligomerized product composition consists essentially of n- and/or iso-alkenes. In another embodiment, the oligomerized product composition consists of n- and/or iso-alkenes. In some implementations, the oligomerized product composition comprises 0 wt % to less than 10 wt % aromatics, such as 0 wt % to less than 5 wt % aromatics. In certain implementations, the oligomerized product composition comprises 0 wt % to less than 3 wt % aromatics. In some embodiments, aromatic hydrocarbons may be formed at a concentration below 4 wt %. In an exemplary implementation, the oligomerized product composition comprises no traces of aromatics.

In some embodiments, the method further comprises hydrogenating the jet-range olefins made according to the described method to produce saturated hydrocarbons suitable for jet fuel.

The disclosed embodiments of process variations offer flexibility to adapt feed variability and are dependent on the efficacy of the core C₂₊ co-oligomerization technology. In some embodiments, more efficient co-oligomerization will lower the need for larger recycle loops and make single-pass processes more viable.

### IV. Catalysts

In some embodiments, the hybrid catalyst system comprises a transition metal-based catalyst and an acid catalyst. A single catalyst component according to the present disclosure can also be a transition metal-based catalyst but does not comprise a separate acid catalyst component.

The transition metal-based catalyst can be a catalyst capable of catalyzing a Cossee-Arlman mechanism of oligomerization. In exemplary embodiments, the transition metal-based catalyst facilitates oligomerizing C₂ olefins present in the feedstock.

In particular embodiments, the transition metal-based catalyst and the single catalyst component, independently, comprise a metal component embedded on a solid support. In certain embodiments, the transition metal-based catalyst and the single catalyst component, independently, comprise nickel (Ni), platinum (Pt), rhodium (Rh), palladium (Pd), or other transition metals, including any combinations and/or alloys thereof. In certain embodiments, the transition metal-based catalyst and the single catalyst component, independently, are a transition metal-loaded aluminosilicate. In some applications, the transition metal-based catalyst and the single catalyst component, independently, are a nickel-loaded aluminosilicate catalyst. In some embodiments, the solid support is silica alumina. In certain embodiments, the solid support is large pore zeolite. In one embodiment, the solid support is amorphous silica alumina.

In some embodiments, the transition metal-based catalyst and the single catalyst component, independently, are obtained via embedding a transition metal on a solid support. In one embodiment, the transition metal-based catalyst is derived by exchanging Ni²⁺ ions onto alumina-silicates to provide Ni-SiAl. The nickel introduction for the transition metal-based catalyst and/or the single catalyst component may be achieved by wetness impregnation, incipient wetness impregnation, or ion exchange. In some embodiments, the transition metal-based catalyst comprises Ni/SiO₂—Al₂O₃. In some embodiments, the single catalyst component comprises a Ni-embedded support wherein the support is selected from a BEA zeolite, a HY zeolite, a MOR zeolite, SiO₂—Al₂O₃, or MCM-41.

The acid catalyst of the hybrid catalyst system can be a catalyst capable of catalyzing Brønsted acid (BA) catalyzed oligomerization. In exemplary embodiments, the acid catalyst facilitates oligomerizing C₃₊ olefins to generate C₈₊ olefins through BA-catalyzed oligomerization. In some embodiments, C₃₊ olefins are oligomerized through the acid catalyst to generate C₆-C₁₈ olefins.

In some embodiments, the acid catalysts of the hybrid catalyst component and/or the support of the single catalyst component can comprise significant Brønsted acid (BA) sites. In some embodiments, the acid catalyst of the hybrid catalyst component and/or the support of the single catalyst component comprises MFI zeolites. In certain embodiments, the acid catalyst of the hybrid catalyst component and/or the support of the single catalyst component comprises MFI zeolites with a Si/Al ratio ranging from 10 to 300, such as from 10 to 290, 10 to 250, 30 to 200, 30 to 280, 40 to 150, 50 to 100, 30 to 80, 60 to 90, or 70 to 90. In one embodiment, the acid catalyst of the hybrid catalyst component and/or the support of the single catalyst component comprises MFI zeolites with a Si/Al ratio ranging from 70 to 90. In another embodiment, the acid catalyst of the hybrid catalyst component and/or the support of the single catalyst component comprises MFI zeolites with a Si/Al ratio of 80.

In some embodiments, the MFI zeolite is ZSM-5 zeolite. In some embodiments, the ZSM-5 zeolite is selected from a zeolite sold by Zeolyst, such as CBV 3014, CBV 5524, CBV 8014 and CBV 28014. In another embodiment, the ZSM-5 zeolite is CBV 8014. In certain embodiment, the acid catalyst comprises a BEA zeolite catalyst.

In some embodiments, the hybrid catalyst system comprises a Ni-based catalyst that also promotes BA sites. In certain embodiments, the hybrid catalyst system comprises a Ni-SiAl catalyst and a separate MFI zeolite catalyst. In one embodiment, the hybrid catalyst system comprises Ni/SiO₂—Al₂O₃ and MFI zeolites with a Si/Al ratio ranging from 10 to 300, such as from 10 to 250, from 30 to 200, from 40 to 150, from 50 to 100, from 60 to 90, and from 70 to 90. In another embodiment, the hybrid catalyst system comprises Ni/SiO₂—Al₂O₃ and MFI zeolites with a Si/Al ratio of 80.

In some other embodiments, the single catalyst component comprises a metal selected from Ni, Cu, or Ag that is embedded on a support selected from a BEA zeolite, a HY zeolite, SiO₂—Al₂O₃, a MOR zeolite, or MCM-41. In some embodiments, the metal is present in an amount ranging from 0.5 wt. % to 10 wt. %, such as 0.5 wt. % to 7 wt. %, or 0.5 wt. % to 1 wt. %. In particular such embodiments, the metal is Ni, and the support is a BEA zeolite. In certain representative embodiments, the metal is Ni, and the Ni is present in an amount of 2 wt. % or 5 wt. %.

### V. Products

The disclosed method provides an oligomerized product composition that comprises jet-range olefins. In some embodiments, the jet-range olefins comprise olefins having carbon numbers ranging from C₆ to C_{16.} In certain embodiments, the jet-range olefins comprise C₈ to C₁₆ olefins. In one embodiment, the jet-range olefins comprise C₈ to C₁₅ olefins. In another embodiment, the jet-range olefins comprise C₁₀ to C₁₅ olefins. In yet another embodiment, the jet-range olefins comprise C₁₀ to C₁₃ olefins.

The oligomerized product composition may further comprise non-jet-range products. In some embodiments, a ratio of the jet-range olefins to the non-jet-range products may be at least 25%.

In some embodiments using the single catalyst component, the ratio of the jet-range olefins to the non-jet-range products ranges from 25% to 90%. In certain embodiments, the ratio of the jet-range olefins to the non-jet-range products ranges from 30% to 70%. In some other embodiments, the ratio of the jet-range olefins to the non-jet-range products ranges from 70% to 85%. In one embodiment, the ratio of the jet-range olefins to the non-jet-range products is at least 30%. In another embodiment, the ratio of the jet-range olefins to the non-jet-range products is 30%. In yet another embodiment, the ratio of the jet-range olefins to the non-jet-range products is 50%.

In some other embodiments, using the hybrid catalyst component, the ratio of the jet-range olefins to the non-jet-range products is at least 30%, such as at least 40%, at least 50%, at least 60%, or at least 65%. In further embodiments, the ratio of the jet-range olefins to the non-jet-range products is at least 70%. In one embodiment, the ratio of the jet-range olefins to the non-jet-range products is at least 75%. In another embodiment, the ratio of the jet-range olefins to the non-jet-range products is at least 80%.

In some other embodiments, using the hybrid catalyst component, the ratio of the jet-range olefins to the non-jet-range products ranges from 70% to 85%.

In some embodiments, at least 40% of the mixture of C₂ and C₃₊ olefins is converted into jet-range olefins in a single pass after a time on stream. In certain embodiments, the time on stream is at least 50 hours. In one embodiment, the time on stream is at least 60 hours.

In other embodiments, at least 80% of the mixture of C₂ and C₃₊ olefins is converted into jet-range olefins in a single pass after a time on stream. In certain embodiments, the time on stream is at least 50 hours. In one embodiment, the time on stream is at least 60 hours.

In yet another embodiment, at least 90% of the mixture of C₂ and C₃₊ olefins is converted into jet-range olefins in a single pass after a time on stream. In certain embodiments, the time on stream is at least 50 hours. In one embodiment, the time on stream is at least 60 hours.

In some embodiments using the single catalyst component with a mix of C₂ and C₄ olefins, at least 20% C₂ olefin is converted into jet-range and/or diesel-range olefins in a single pass after a time on stream. In some examples, at least 40% C₂ olefin is converted. In certain embodiments, from 25% to 80% C₂ olefin is converted into jet-range and/or diesel-range olefins in a single pass after a time on stream. In one embodiment, from 40% to 75% C₂ olefin is converted. In another embodiment, from 65% to 70% C₂ olefin is converted.

In some other embodiments using the single catalyst component with a mix of C₂ and C₄ olefins, from 40% to 85% C₄ olefin is converted into jet-range and/or diesel-range olefins in a single pass after a time on stream. In certain embodiments, from 45% to 80% C₄ olefin is converted. In one embodiment, from 50% to 75% C₄ olefin is converted. In another embodiment, from 65% to 70% C₄ olefin is converted.

In yet other embodiments using the single catalyst component with a mix of C₂ and C₄ olefins, at least 40% C₄ olefin is converted into jet-range and/or diesel-range olefins in a single pass after a time on stream. In certain embodiments, at least 45% C₄ olefin is converted. In one embodiment, at least 50% C₄ olefin is converted. In another embodiment, at least 65% C₄ olefin is converted.

In some embodiments using the single catalyst component, at least 60% of the mixture of C₂ and C₄ olefins is converted into jet-range and/or diesel-range olefins in a single pass without recycling. In certain embodiments, at least 70% of the mixture of C₂ and C₄ olefins is converted into jet-range and/or diesel-range olefins in a single pass without recycling.

### VI. Reactor and System Embodiments

Embodiments of a reactor and a system for converting a feedstock comprising mixture of C₂ and C₃₊ olefins into jet-range and, in some particular embodiments also diesel-range, olefins via co-oligomerization are disclosed herein. In some embodiments, the system comprises the reactor along with other system components.

In some embodiments, the reactor comprises a hybrid catalyst system or a single catalyst component as described herein. In certain embodiments, the hybrid catalyst system comprises a transition metal-based catalyst and an acid catalyst. In one embodiment, the transition-metal based catalyst and the acid catalyst are provided on separate catalyst beds. In another embodiment, the transition-metal based catalyst and the acid catalyst are provided on the same catalyst bed. In embodiments using a single catalyst component, the single catalyst component is provided on a single catalyst bed within the reactor.

In any of the foregoing or following embodiments, the hybrid catalyst system is provided in an amount sufficient to convert C₂ and C₃₊ olefins into jet-range olefins via co-oligomerization. In other embodiments, the single catalyst component is provided in an amount sufficient to convert C₂ and C₄ olefins into jet-range and/or diesel-range olefins via co-oligomerization.

In some embodiments, the system comprises one or more storage containers, wherein each storage container independently contains a different olefin feedstock, such as a C₂ olefin feedstock, a C₃₊ olefin feedstock, or combinations thereof. For example, in some embodiments, one storage container can house C₃ olefins, a second storage container can house C₄ olefins, a third storage container can house C₅ olefins, and a fourth storage contain can house C₂ olefins. In yet other embodiments, a single storage container can house a mixture of C₃₊ olefins (e.g., a mixture of C₃, C₄, and C₅ olefins), or a mixture of C₂ and C₃₊ olefins. Each storage container typically is maintained at a pressure lower than a vapor pressure of the olefin housed within the storage container.

In some embodiments, the system further comprises a high-pressure syringe pump manifold. The high-pressure syringe pump manifold can comprise one or more syringes that are configured to direct feedstocks housed in the storage containers into the reactor. In some embodiments comprising a plurality of syringes, the syringes are arranged in parallel configuration. In such embodiments, each syringe of the plurality can be independently and fluidly coupled to a storage container housing a feedstock component (e.g., storage containers housing C₂ olefins, C₃₊ olefins, or combinations thereof).

In some embodiments, the reactor is connected to a storage container comprising a feedstock source comprising a mixture of C₂ and C₃₊ olefins. In some other embodiments, the reactor is connected to a first storage container comprising the feedstock source comprising the C₂ olefins and a second storage container comprising the feedstock source comprising the C₃₊ olefins. In one embodiment, the reactor is connected to a first storage container comprising feedstock source comprising C₃ olefins, a second storage container comprising a feedstock source comprising C₄ olefins, a third storage container comprising a feedstock source comprising C₅ olefins, and a fourth storage container comprising a feedstock source comprising C₂ olefins. In another embodiment, the reactor is connected to a first storage container comprising a feedstock source comprising C₄ olefins and a second storage container comprising a feedstock source comprising C₂ olefins. In some embodiments, the reactor is further connected to the high-pressure syringe pump manifold as described herein.

In some embodiments, the reactor is maintained at a pressure higher than the vapor pressure of the C₃₊ olefins during operation.

In some embodiments, the reacting pressure of the reactor ranges from 50 psig to 500 psig, such as 50 psig to 450 psig, or 55 psig to 400 psig, or 60 psig to 400 psig, or 70 psig to 350 psig, or 75 psig to 350 psig, or 75 psig to 300 psig, or 75 psig to 200 psig, or 75 psig to 150 psig, or 75 psig to 125 psig. In some embodiments, the pressure can range from 200 psig to 500 psig. In certain embodiments, the olefins are oligomerized under a pressure ranging from 75 to 500 psig, such as from 75 to 300 psig, or 75 psig to 200 psig. In one embodiment, the pressure is 100 psig. In another embodiment, the pressure is 200 psig.

In certain embodiments, the reacting pressure of the reactor ranges from 75 psig to 300 psig, such as 75 psig to 250 psig, or 75 psig to 200 psig, or 75 psig to 150 psig, or 75 psig to 120 psig, or 75 psig to 110 psig, or 75 psig to 100 psig. In particular embodiments, the reactor is operated at a pressure of 100 psig. In another embodiments, the reactor is operated at a pressure of 200 psig.

In some other embodiments, such as embodiments using a single catalyst component, the reacting pressure of the reactor can range from 75 psig to 500 psig, such as 80 psig to 500 psig, or 100 psig to 520 psig, or 100 psig to 500 psig, or 200 psig to 500 psig, or 75 psig to 300 psig, or 150 psig to 250 psig, or 160 psig to 240 psig, or 170 psig to 230 psig, or 180 psig to 220 psig, or 190 psig to 210 psig. In particular embodiments, the reactor is operated at a pressure of 200 psig. In another embodiments, the reactor is operated at a pressure of 100 psig.

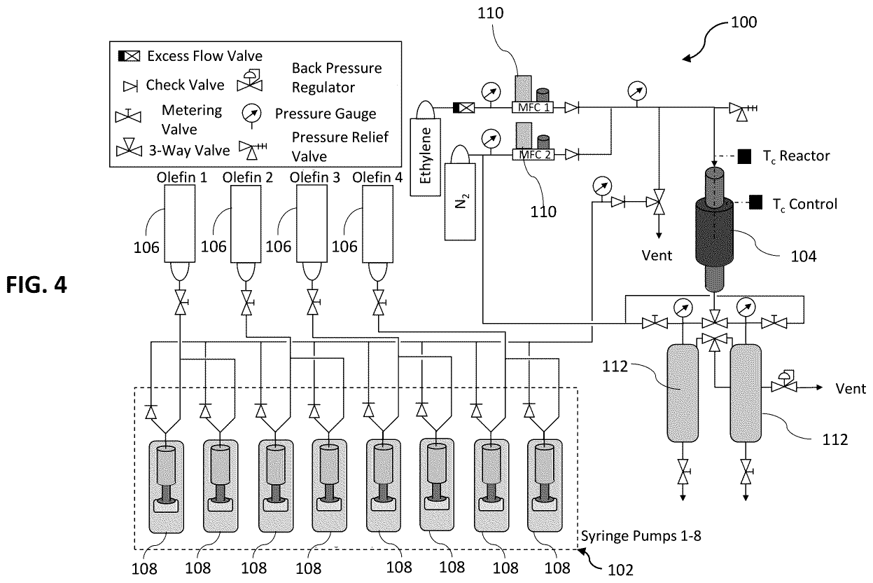

FIG. 4 shows a simplified schematic of an embodiment of a reactor system 100 used for method embodiments disclosed herein. In some embodiments, the system comprises high-pressure syringe pump manifold 102 to pump liquefied olefins to reactor 104. In such embodiments, the liquefied olefins are stored in cylinders 106 under their own vapor pressure. In such embodiments, reactor 104 is operated at pressures higher than each individual cylinder pressure. In further embodiments, each olefin is pumped by two parallel syringes 108 to reduce pulsing in the feed flowrates. In some embodiments, the system can further comprise mass flow controllers (e.g., MFCs 110 in FIG. 4), which are used to control feed flowrates.

In some embodiments, the method is conducted using a down-flow reactor arrangement. In such embodiments, the hybrid catalyst system is placed in the reactor, typically in an isothermal zone, and heated using a band heater to the desired reaction temperature. In some embodiments, the reactor comprises a single hybrid catalyst bed comprising a transition metal-based catalyst (e.g., Ni/SiO₂—Al₂O₃ catalyst) and a zeolite catalyst, wherein the transition metal-based catalyst is positioned towards the top of the reactor, which is fluidly connected to the feedstock source container(s) such that the feedstock is first exposed to the transition metal-based catalyst; and wherein the zeolite catalyst is positioned towards the bottom of the reactor. In such embodiments, the feedstocks and a carrier gas (e.g., N₂) are fed from the top of the reactor, and the flow rates of these gaseous feedstocks are controlled by mass flow controllers. In certain embodiments, liquid products can be collected at the bottom of the reactor in a cold trap (e.g., traps 112 in FIG. 4). In embodiments utilizing a single catalyst component, the catalyst can be positioned in a catalyst bed within the reactor.

### VII. Method Embodiments

Embodiments of methods for using a system for converting a feedstock comprising mixture of C₂ and C₃₊ olefins into jet-range olefins via co-oligomerization also are disclosed herein.

In some embodiments, the method comprises contacting, in a single reactor as described herein, a feedstock comprising a mixture of C₂ and C₃₊ olefins (e.g., C₂ and C₄ olefins) with a single catalyst component, and heating the single reactor at a temperature ranging from 100° C. to 500° C. to convert the feedstock into an oligomerized product composition comprising jet-range and/or diesel-range olefins via co-oligomerization. In certain embodiments, the single reactor is operated at a temperature ranging from 100° C. to 500° C., such as 150° C. to 500° C., or 200° C. to 500° C., or 250° C. to 500° C., or 250° C. to 400° C., or 250° C. to 300° C. In some embodiments, the temperature can range from 150° C. to 300° C. In one embodiment, the method comprises heating the single reactor at a temperature ranging from 250° C. to 300° C. to convert the feedstock into an oligomerized product composition comprising jet-range and/or diesel-range olefins via co-oligomerization.

In some embodiments, the method comprises contacting, in a single reactor as described herein, a feedstock comprising a mixture of C₂ and C₃₊ olefins with a hybrid catalyst system comprising a transition-metal based catalyst and an acid catalyst, and heating the single reactor at a temperature ranging from 100° C. to 500° C. to convert the feedstock into an oligomerized product composition comprising jet-range olefins via co-oligomerization. In certain embodiments, the single reactor is operated at a temperature ranging from 100° C. to 500° C., such as 150° C. to 500° C., or 200° C. to 500° C., or 250° C. to 500° C., or 250° C. to 400° C., or 250° C. to 300° C., or 150° C. to 300° C. In one embodiment, the method comprises heating the single reactor at a temperature ranging from 250° C. to 500° C. to convert the feedstock into an oligomerized product composition comprising jet-range olefins via co-oligomerization. In another embodiment, the method comprises heating the single reactor at a temperature ranging from 250° C. to 300° C. to convert the feedstock into an oligomerized product composition comprising jet-range olefins via co-oligomerization.

In some embodiments, the method includes introducing the C₂ olefin into the reactor, introducing the C₃₊ olefins into the reactor, contacting the mixture of C₂ and C₃₊ olefins with the hybrid catalyst system (e.g., by allowing the feedstock to flow through the reactor and thus become exposed to the hybrid catalyst system), and heating the reactor to convert the mixture of C₂ and C₃₊ olefins into an oligomerized product composition comprising jet-range olefins. In some embodiments, the oligomerized product composition further comprises non-jet-range products comprising olefins having carbon numbers lower than 8. In such embodiments, the reactor is heated at a temperature ranging from 100° C. to 500° C. In some embodiments, the single reactor is heated at a temperature ranging from 100° C. to 500° C., such as 150° C. to 500° C., or 250° C. to 500° C., or 250° C. to 300° C., or 150° C. to 450° C., or 150° C. to 400° C., or 150° C. to 350° C., or 150° C. to 300° C. In certain embodiments, the single reactor is heated at a temperature ranging from 250° C. to 500° C., such as 250° C. to 450° C., or 250° C. to 400° C., or 250° C. to 350° C., or 250° C. to 300° C. In one embodiment, the single reactor is heated at a temperature ranging from 250° C. to 300° C.

In further embodiments, the method comprises separating the jet-range olefins from the non-jet-range products and contacting the non-jet-range products with the hybrid catalyst system to produce jet-range olefins, such as by recycling the non-jet-range products back into the reactor.

In some other embodiments, the method comprises contacting, in a single reactor as described herein, a feedstock comprising a mixture of C₂ and C₄ olefins with a single catalyst component and heating the single reactor at a temperature ranging from 100° C. to 500° C. to convert the feedstock into an oligomerized product composition comprising jet-range and/or diesel-range olefins via co-oligomerization. In certain embodiment, the single reactor is heated at a temperature ranging from 100° C. to 500° C., such as 100° C. to 450° C., or 100° C. to 400° C., or 150° C. to 500° C., or 250° C. to 500° C., or 250° C. to 300° C., or 150° C. to 350° C., or 150° C. to 300° C. In certain embodiments, the single reactor is heated at a temperature ranging from 150° C. to 280° C., such as 160° C. to 280° C., or 180° C. to 280° C., or 200° C. to 260° C., or 210° C. to 250° C., or 215° C. to 245° C., or 220° C. to 240° C. In one embodiment, the single reactor is heated at a temperature ranging from 225° C. to 235° C.

In further embodiments, the method comprises separating the jet-range olefins from the non-jet-range products and contacting the non-jet-range products with the single catalyst system to produce jet-range olefins, such as by recycling the non-jet-range products back into the reactor.

### VIII. Combination Embodiments

Also described herein are embodiments of a combination comprising a feedstock as described herein and a catalyst system. In some embodiments, the feedstock of the combination comprises a mixture of olefins. In certain embodiments, the mixture of olefins comprises a mixture of C₂ and C₃₊ olefins.

In some embodiments, the catalyst system is a single catalyst component, and the feedstock comprises a mixture of C₂ and C₄ olefins. In exemplary embodiments, the single catalyst component is a Ni-promoted BEA zeolite catalyst.

In some other embodiments, the catalyst system is a hybrid catalyst system. In further embodiments, the hybrid catalyst system of the combination is the hybrid catalyst system as described herein. In certain embodiments, the hybrid catalyst system of the combination comprises a transition metal-based catalyst and an acid catalyst. In one embodiment, the transition-metal based catalyst comprises Ni/SiO₂—Al₂O₃, the acid catalyst comprises ZSM-5 zeolites having a Si/Al ratio ranging from 10 to 300.

### IX. Overview of Several Embodiments

Disclosed herein are embodiments of a method, comprising: contacting, in a single reactor, a feedstock comprising a mixture of C₂ and C₃₊ olefins with a hybrid catalyst system comprising a transition-metal based catalyst and an acid catalyst; and heating the single reactor at a temperature ranging from 250° C. to 500° C. to convert the feedstock into an oligomerized product composition comprising jet-range olefins via co-oligomerization.

The any or all embodiments, the oligomerized product composition further comprises non-jet-range products.

In any or all of the above embodiments, the method further comprising separating the jet-range olefins from the non-jet-range products; and/or contacting the non-jet-range products with the hybrid catalyst system to produce jet-range olefins.

In any or all of the above embodiments, contacting the feedstock with the hybrid catalyst system comprises (i) contacting the feedstock with the transition-metal based catalyst, which is provided in a first catalyst bed contained within the single reactor to produce a first product mixture and then contacting the first product mixture with the acid catalyst, which is provided in a second catalyst bed contained within the single reactor to convert the first product mixture into the oligomerized product composition; or (ii) contacting the feedstock with the transition-metal based catalyst and the acid catalyst, wherein the transition-metal based catalyst and the acid catalyst are provided on the same catalyst bed in the single reactor.

In any or all of the above embodiments, the oligomerized product composition comprises 0 wt % to less than 5 wt % aromatics.

In any or all of the above embodiments, the feedstock is derived from a heterogeneous carbonaceous feed source via methanol synthesis and a methanol-to-olefin process.

In any or all of the above embodiments, the feedstock comprises mixed olefins with a C₃₊:C₂ molar ratio ranging from 5:1 to 1:5.

In any or all of the above embodiments, the C₃₊ olefins comprise olefins having a carbon number of C₃, C₄, C₅, or a combination of such olefins.

In any or all of the above embodiments, the jet-range olefins comprise olefins having carbon numbers ranging from C₈ to C₁₆.

In any or all of the above embodiments, the temperature ranges from 250° C. to 300° C.

In any or all of the above embodiments, the method is carried out under a pressure ranging from 75 psig to 300 psig.

In any or all of the above embodiments, the transition-metal based catalyst comprises nickel (Ni).

In any or all of the above embodiments, the transition-metal based catalyst is Ni/SiO₂—Al₂O₃.

In any or all of the above embodiments, the acid catalyst comprises a ZSM-5 zeolite having a Si/Al ratio ranging from 10 to 250.

In any or all of the above embodiments, the method provides a single pass conversion of the mixture of C₂ and C₃₊ olefins into jet-range olefins of at least 90% after a time on stream of at least 60 hours.

In any or all of the above embodiments, a ratio of the jet-range olefins to the non-jet-range products is at least 65%.

Also disclosed are embodiments of a reactor for performing the method according to any or all of the above embodiments, the reactor comprising a hybrid catalyst system comprising a transition-metal based catalyst and an acid catalyst, wherein the transition-metal based catalyst and the acid catalyst are provided on separate catalyst beds or on the same catalyst bed and the hybrid catalyst system is provided in an amount sufficient to convert C₂ and C₃₊ olefins into jet-range olefins via co-oligomerization.

In any or all of the above embodiments, the reactor is further connected to (i) a feedstock source comprising the mixture of C₂ and C₃₊ olefins or a first feedstock source comprising the C₂ olefins and a second feedstock source comprising the C₃₊ olefins; (ii) a high-pressure syringe pump manifold comprising one or more syringes; or (iii) a combination of (i) and (ii).

Also disclosed are embodiments of a combination, comprising a feedstock comprising a mixture of C₂ and C₃₊ olefins; and a hybrid catalyst system comprising a transition-metal based catalyst and an acid catalyst.

In any or all of the above embodiments, the transition-metal based catalyst comprises Ni/SiO₂—Al₂O₃, the acid catalyst comprises ZSM-5 zeolites having a Si/Al ratio ranging from 10 to 250.

Also disclosed are embodiments of a method, comprising contacting, in a single reactor, a feedstock comprising a mixture of C₂ and C₄ olefins with a single catalyst component comprising a transition-metal based catalyst and an acid catalyst; and heating the single reactor at a temperature ranging from 150° C. to 300° C. to convert the feedstock into an oligomerized product composition comprising jet-range and/or diesel-range olefins via co-oligomerization.

### X. Examples

### Examples 1-11

All experiments were performed in a packed bed reactor system. Nickel introduction into catalyst embodiments was achieved by wetness impregnation, incipient wetness impregnation, or ion exchange. Several pretreatment strategies were also evaluated, including both pre-reduction in hydrogen and thermal treatment in flowing nitrogen.

Baseline catalyst preparation—The baseline catalyst was prepared via ion exchange of an amorphous silica alumina catalyst (Davicat SiAl 3113) with nickel chloride via the following procedure: The silica alumina support was calcined at 550° C. prior to ion exchange. Ion exchange was achieved by heating a stirred mixture of the silica alumina support, NiNO₃ and water to 90° C. under reflux for 24 hours. After ion exchange the catalyst was washed thoroughly with warm deionized water and dried at 90° C. overnight. The dried catalyst was then pelletized and loaded into the flow reactor system. Prior to reaction, the catalyst was pretreated at 300° C. in flowing N₂ at atmospheric pressure before cooling to the reaction temperature.

Product Analysis—products were analyzed via gas chromatography—mass spectroscopy (GC-MS) and quantified with a flame ionization detector (FID). The gaseous products were passed through a flow meter (DryCal) and their composition was analyzed in a gas chromatography—thermal conductivity detector (GC-TCD). Although the typical temperature range in which the experiments were performed are 200-300° C., most of the oligomerization experiments were carried out 275° C. The typical reaction pressure used herein was 100 psig with a weigh hourly space velocity 0.6-1.8 h⁻¹.

Preparation of Hybrid/Catalyst—Ni/SiO₂—Al₂O₃ catalyst was prepared via ion exchange of an amorphous silica alumina catalyst (Davicat SiAl 3113) with NiNO₃ via the following procedure.

The silica alumina support was calcined at 550° C. prior to ion exchange. Ion exchange was achieved by heating a stirred mixture of the silica alumina support, NiNO₃ and water to 98° C. under reflux for 24 hours. After ion exchange the catalyst was washed thoroughly (5-6 times) with warm deionized water and dried at 90° C. overnight. The dried catalyst was then pelletized and loaded into the flow reactor system. Prior to reaction the catalyst was pretreated at 300° C. in flowing N₂ at atmospheric pressure. The typical Ni loading used in this catalyst was 2 wt % which was determined by inductively coupled plasma atomic emission spectroscopy (ICP-AES). Different zeolite catalysts used in this example were commercially available and obtained from Zeolyst Internationals. Prior to loading in the reactor, all the zeolite materials were pelletized and calcined at 550° C. for 4 hours under air.

### Example 1

In this example, ethylene oligomerization over a baseline Ni-SiAl catalyst was evaluated at a temperature of 150° C. and pressure of 300 psig. Ethylene oligomerization was performed at 150° C. and 300 psig. The ethylene (and other olefins as appropriate) conversion was monitored via online gas chromatography and liquid products were sampled every 24 hours.

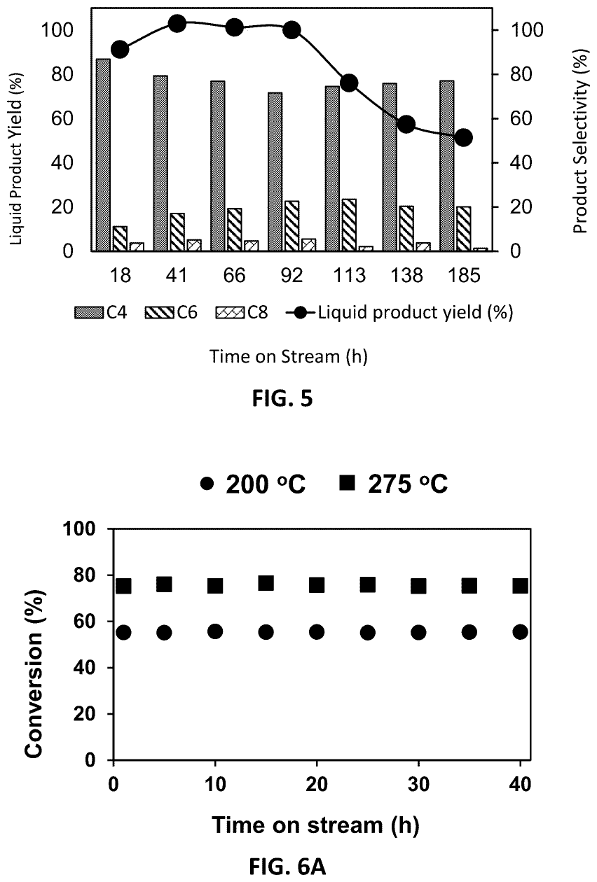

FIG. 5 shows the performance of the baseline Ni—SiAl catalyst corresponding to ethylene oligomerization. At the initial conditions, the baseline catalyst exhibits nearly complete ethylene conversion for 100 hours time on stream before slowly deactivating. The product selectivity towards larger olefins varied little with time on stream. All produced olefins had even carbon numbers, butenes (C₄) and hexenes (C₆) being the majority products. In this particular example, the presence of the catalyst (after the 200-hour evaluation) suggested deactivation is likely largely due to the buildup of carbon deposits.

### Example 2

In this example, propylene oligomerization over a Ni/SiAl catalyst at 200° C. was evaluated. Nearly 60% of the propylene conversion was noted at this condition and no deactivation was observed for 24 hours. The liquid obtained as the product was analyzed by GC which shows the presence of C₆ and C₉ compounds predominantly with selectivity to 66% and 28%, respectively. A minor amount of C₁₂ was also noted in the product stream.

### Example 3

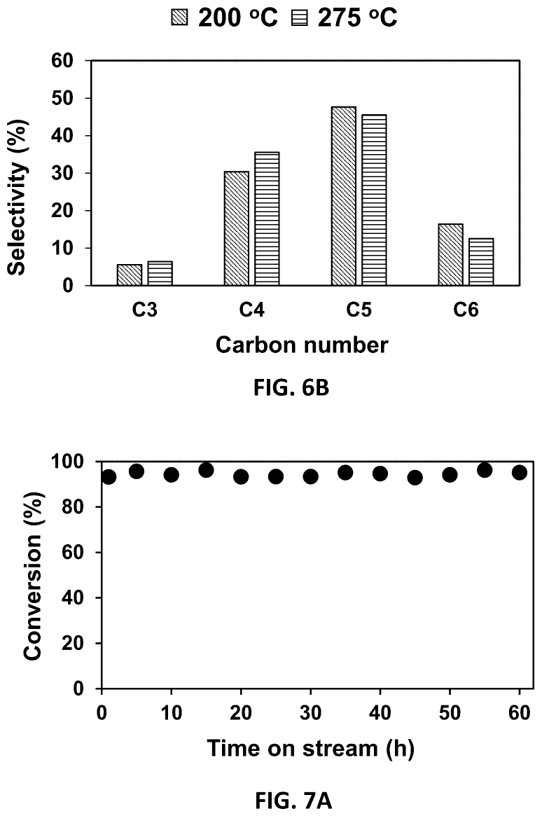

In this example, the co-oligomerization of ethylene/propylene on Ni/SiO₂—Al₂O₃ catalyst at 200° C. and 275° C. was evaluated. Co-oligomerization of a mixture of ethylene and propylene (1:1 molar ratio) was initially carried out at 150° C., the temperature used for baseline ethylene oligomerization; however, only butene was obtained as a product, which suggested that propylene was not reactive at this temperature. When the temperature was increased to 200° C., a significant amount of cross-oligomerization between C₂ and C₃ was noted which yielded C₅ as the major product with selectivity to 48%. In addition to the cross-oligomerization, self-oligomerization of ethylene and propylene was also noted which resulted in different C₄ (30% selectivity) and C₆ (16% selectivity) as the products, respectively. In this particular example, nearly 3 times higher selectivity to C₅ compared to C₆ further indicated that majority of the propylene underwent cross oligomerization with ethylene compared to reacting with itself. The overall conversion of C₂ and C₃ was noted at 55%. The conversion and selectivity of the product remained same ever after 40 hours after which the overall conversion decreased by 10%. Upon increasing the reaction temperature to 275° C., the overall conversion of C₂ and C₃ was further increased to 75%. The analysis of the liquid sample obtained as the product shows very similar to the product distribution noted at 200° C. In this particular example, the results suggested that although increasing temperature significantly influence the activity of co-oligomerization, it merely affected the product distribution. FIGS. 6A and 6B show the time on stream (TOS) data (FIG. 6A) and selectivity to different products (FIG. 6B) respectively corresponding to co-oligomerization of ethylene/propylene over Ni/SiAl catalyst at both 200° C. and 275° C.

In this particular example, liquid products accounted for 95% of mass balance and thus suggests little to no over-oligomerization in all these cases. While the co-oligomerization of C₂ and C₃ olefin (stability>40 hours on TOS) with product selectivity to 60-65% for C₅₊ products has been demonstrated in this particular example, it mostly comprised of C₅ and C₆.

### Example 4

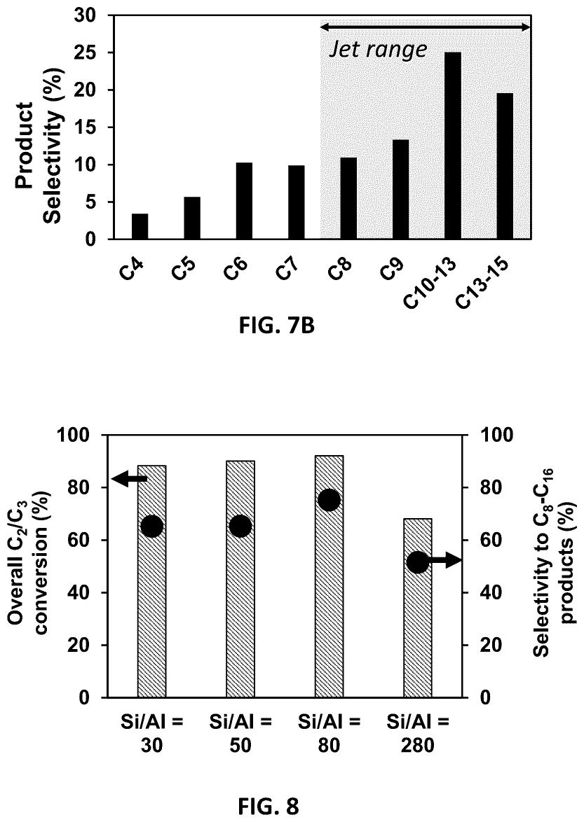

In this example, the co-oligomerization of ethylene/propylene using a hybrid catalyst system containing Ni/SiO₂—Al₂O₃ and zeolite catalyst at 275° C. was evaluated. A hybrid catalyst bed was used, which included the Nickel catalyst and the ZSM-5 zeolite to promote an acid catalyzed oligomerization pathway and enable C₅₊ olefins (that are generated in-situ upon oligomerization of C₂ and C₃ on Ni catalyst) to oligomerize further to generate higher olefins suitable for jet range. In this example, when a mixture of ethylene and propylene was reacted at 275° C. using the hybrid catalyst system, the majority of products obtained were in the C₁₀-C₁₅ range. Using the hybrid catalyst bed not only improved the selectivity to desired products but also increased the single pass conversion to 90-95%. FIG. 7A shows the time on stream data which shows no sign of deactivation for nearly 60 hours. FIG. 7B shows the product selectivity resulted from the co-oligomerization which shows 70% of the products are distributed in the range of C₈-C₁₅ with a peak residing around C₁₀-C₁₃, typical for aviation fuel. Analysis of GC-MS of the product stream confirmed that all the products obtained in this example comprised n- or iso-alkenes and no traces of aromatics were noted in the product stream. In this particular example, based on the product selectivity obtained herein (FIG. 7B), 30% of the products are still in the range of C₃ to C₇. In some embodiments, these products can be recycled to the same catalyst bed to undergo further oligomerization process and improve the overall efficiency of the process.

### Example 5

In this example, co-oligomerization of mixed olefins as described above in Example 4 was performed with similar conditions but using a stage bed configuration in which a Ni/SiO₂—Al₂O₃ catalyst bed and the ZSM-5 catalyst bed were placed consecutively. This configuration of the reactor allowed the olefins (i.e., ethylene and propylene) to react with the Ni/SiO₂—Al₂O₃ first and the resultant product (i.e., C₄-C₆) then further reacts with ZSM-5 which resulted in products with longer carbon chain length (typical range C₈-C₁₆).

### Example 6

In this example, oligomerization in a mixed bed configuration was also carried out wherein the amounts of both Ni/SiO₂—Al₂O₃ and ZSM-5 catalyst were mixed together first and then placed in the reactor.

### Example 7

In this example, the impact of acidity of zeolites catalyst on the co-oligomerization of ethylene and propylene was evaluated. Co-oligomerization of C₂ and C₃ was evaluated with different ZSM-5 catalysts with varying Si/Al ratio (i.e., varying Bronsted acidity). In this example, as demonstrated in FIG. 7, the presence of acid catalyst can be used to improve both conversion and selectivity to jet range products. Commercially available ZSM-5 materials, such as CBV 3014 (Si/Al ratio 30), CBV 5524 (Si/Al ratio 50), CBV 8014 (Si/Al ratio 80), and CBV 28014 (Si/Al ratio 280) was obtained from Zeolyst International was used in this example. Bronsted acidity increases with decreasing Si/Al ratio. FIG. 8 shows the activity comparison of the different catalysts comprised of Ni/SiO₂—Al₂O₃ and various ZSM-5 catalysts. The effects of Bronsted acidity of the zeolites evaluated in this example can be seen in FIG. 8. A single pass conversion of C₂ and C₃ resulted in a yield close to 90% with the zeolite containing higher acidity such as Si/Al ratio ranging from 30 to 80. 65% single pass conversion was obtained when a catalyst with very high Si/Al ratio (that is, lower Bronsted acid sites) was used.

The total product selectivity akin to jet range (e.g., C₈-C₁₆) was also analyzed and demonstrated in FIG. 8. As with conversion, selectivity to desired product was also higher in the case of zeolites with higher acidity. While the product selectivity was in the range of 65-75% with the zeolite containing Si/Al ratio ranging from 30-80, 50% product selectivity was obtained when zeolites high Si/Al ratio was used. While the product selectivities obtained with CBV 3014, CBV 5524, and CBV 8014 were within 10%, an increase in the product selectivity was obtained with CBV8014.

### Example 8

In this example, the effect of other zeolites with different structures and pore size on oligomerization activity was evaluated. Mordenite (CBV 21A), a one-dimensional zeolite shows an overall conversion 80%, but with a low amount of products in jet fuel range in particular embodiments. Without being limited to a single operating theory, it currently is believed that any low mass balance obtained in particular example may result from formation of polymeric products. Faujasite (CBV 901), a commercially available zeolite with 3D pore structures similar to ZSM-5 albeit larger pore size, also was evaluated. 72% overall conversion was observed in this example.

### Example 9

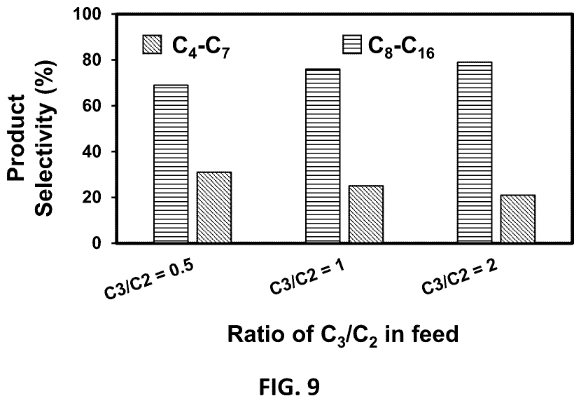

In this example, upon optimization of both Ni²⁺ (Ni/SiO₂—Al₂O₃ ) and acid catalyst (ZSM-5 zeolite, CBV 8014), the impact of ethylene and propylene composition in the feed on the corresponding product selectivity was evaluated. Different mixtures with varying molar ratios of C₃/C₂, such as 2, 1 and 0.5 were prepared and used as feed. When subjected to oligomerization with the hybrid catalyst at 275° C., all these different feeds composition resulted in very similar product spectrum that are composed of different compounds with different carbon numbers which are typically in the range between C₄-C₁₆. Product selectivity corresponding to the jet range i.e., C₈-C₁₆ was further calculated from GC-FID. FIG. 9 shows the C₈-C₁₆ product selectivity obtained with different C₃/C₂ molar ratio.

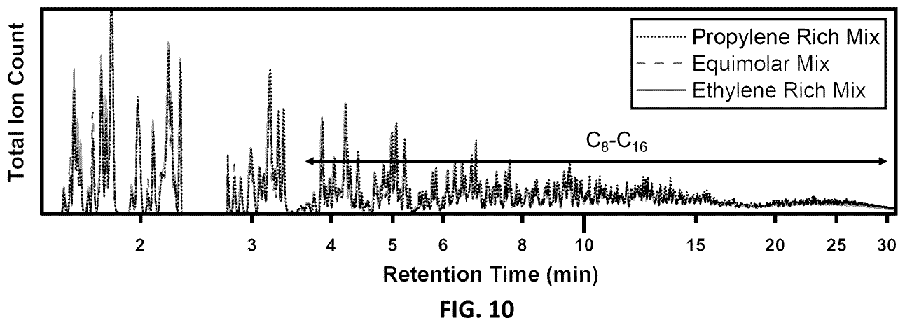

In this example, it is evident from the FIG. 9 that variation in olefin composition can have a subtle effect on the product selectivity. For example, the product selectivity corresponding to C₈-C₁₆ are mostly in the range of 60-70%, whereas lighter products are limited to 30-35%. Similar product selectivity obtained herein despite wide variation of C₂/C₃ composition suggests that at the present reaction conditions the product distribution are governed by equilibrium. FIG. 10 shows the comparison of the GCMS spectra obtained upon oligomerization of C₂/C₃ with varying composition which demonstrates very similar product distribution irrespective of their feed composition.

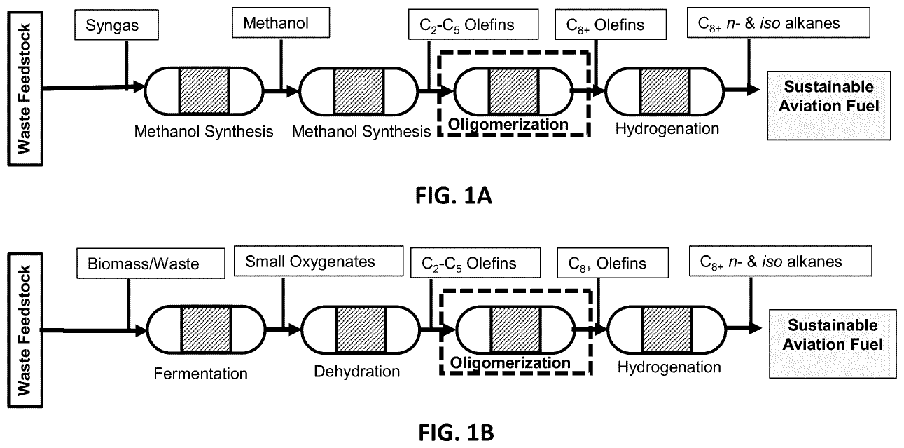

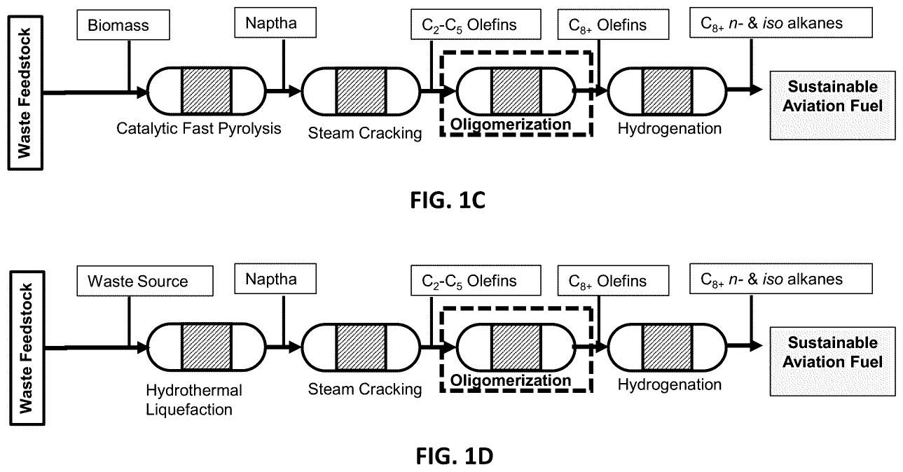

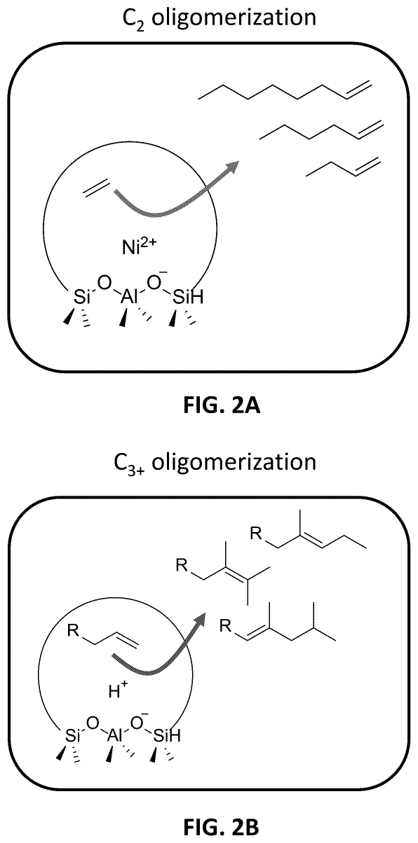

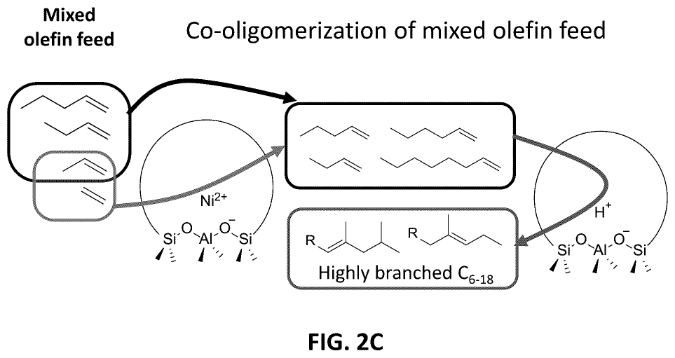

In this example, the results demonstrate the ability of the current process to oligomerize a wide variety of feeds while still resulting in a product suitable for jet fuel. As shown in FIG. 1, mixed olefins with variety of compositions are expected depending on the upstream process and thus, developing a downstream oligomerization process is important to upgrading to sustainable aviation fuel. In this particular example, although the product distribution was similar, it is apparent that a continuous increase in product selectivity corresponding to C₈-C₁₆ was noted with increasing propylene content in the feed. 10% increase in product selectivity was noted when C₃/C₂ ratio was increased from 0.5 to 2.

Propylene oligomerization at the same reaction condition was carried out using Ni/SiO₂—Al₂O₃ and MFI catalyst. Although wide distribution of products (C₄-C₁₆) was obtained, a significant amount of aromatics species such as xylene or naphthalene derivatives were noted. In this example, absence of these species during co-oligomerization suggests that the underlying reaction mechanism corresponding to cross-oligomerization between C₂ and C₃ are very different such that it prevented the formation of aromatics.

### Example 10

In this example, the impact of different feedstock compositions and different temperatures on the corresponding product selectivity was evaluated using a hybrid catalyst comprising Ni/SiO₂—Al₂O₃ and ZSM-5 zeolite. Feedstocks with different compositions (C₂-C₃ with a molar ratio C₂:C₃=1:1, and C₂ -C₅ with a molar ratio C₂:C₃:C₄:C₅=1:1:1:1) were subjected to oligomerization under different temperatures (250° C. and 275° C.). The operation pressure was 100 psig and the WHSV (weight-hourly space velocity) was 0.8 h⁻¹.

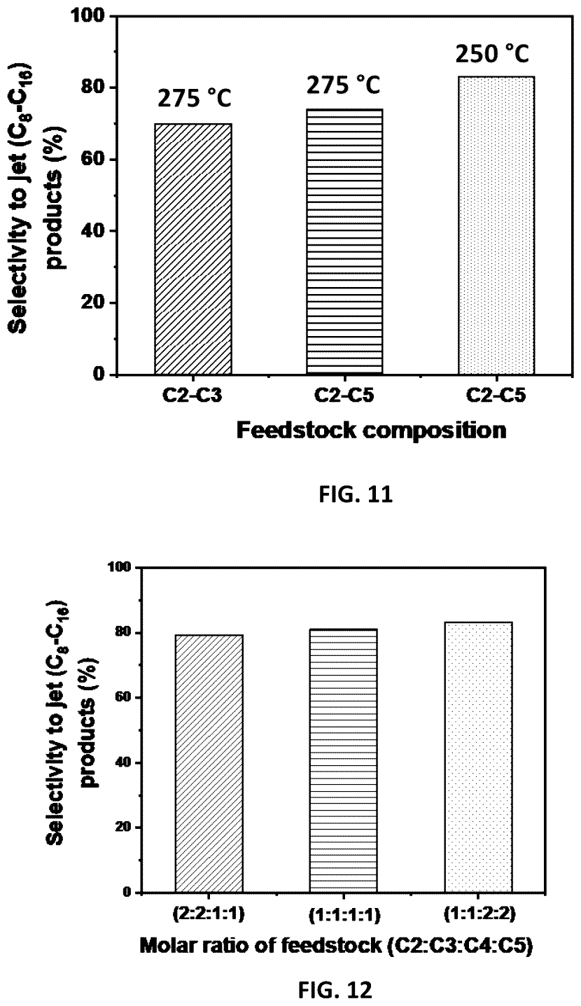

FIG. 11 shows the C₈-C₁₆ product selectivities obtained with the different olefin compositions under different temperatures. The feedstock with a C₂ -C₃ composition oligomerized at 275° C. exhibited a product selectivity of 70.1%. The feedstock with a C₂ -C₅ composition oligomerized at 275° C. exhibited a product selectivity of 74.3%. The feedstock with a C₂ -C₅ composition oligomerized at 250° C. exhibited a product selectivity of 79.9%.

### Example 11

In this example, the impact of feedstock composition with different molar ratios of C₂:C₃:C₄:C₅ on the corresponding product selectivity was evaluated using a hybrid catalyst comprising Ni/SiO₂—Al₂O₃ and ZSM-5 zeolite under the temperature of 250° C. The operation pressure was 100 psig and the WHSV was 0.8 h⁻¹.

FIG. 12 shows the C₈-C₁₆ product selectivities obtained with the different olefin compositions. The feedstock with a C₂-C₅ composition having a molar ratio of C₂:C₃:C₄:C₅=2:2:1:1 exhibited a product selectivity of 78.6%. The feedstock with a C₂-C₅ composition having a molar ratio of C₂:C₃:C₄:C₅=1:1:1:1 exhibited a product selectivity of 79.9%. The feedstock with a C₂-C₅ composition having a molar ratio of C₂:C₃:C₄:C₅=1:1:2:2 exhibited a product selectivity of 82.3%.

Examples 1-11 establish an efficient process that can be used to oligomerize mixed olefin feedstocks in a single reactor unit that uses hybrid catalyst system comprised of Ni/SiAl and MFI zeolite was demonstrated. 90% single pass conversion of C₂ and C₃ was obtained with 70% product selectivity that are distributed in the C₈-C₁₅ range, ideal for aviation fuel. In the examples, the products obtained are comprised of n- or iso-alkenes and no traces of aromatics were observed in the product stream. The remaining 30% products which mostly included C₃-C₇ olefins would be recycled to undergo further oligomerization and improve the efficiency of the overall catalytic process.

### Examples 12-14

Solid Acid Catalyst Synthesis—Solid acid catalysts evaluated included commercially available materials including BEA, H—Y, H-ZSM-5 from Zeolyst. The zeolite powder was pelletized using a pneumatic laboratory press (2-ton pressure force for 1 minute) before being crushed and sieved to the desired particle size (60 to 100 mesh). Before loading the reactor, the zeolites were calcined in a muffle furnace at 500° C. for 4 hours. Ni was impregnated onto support materials via incipient wetness with a Cu nitrate precursor solution dissolved in deionized water. After impregnation, the catalysts were dried at 120° C. for 8 hours and calcined at 500° C. for 4 hours.

Catalytic Activity—Performance evaluations for the oligomerization catalysts were performed in a single fixed-bed reactor. A liquid feed of olefins was transported with an ISCO syringe pump and mixed with a flow of N₂ prior to being fed into the reactor. WHSV (weight-hourly space velocity) was calculated based on the mass flow of olefins per weight of catalyst in an hourly basis (i.e., g_{feed} g_{cat}⁻¹ h⁻¹). All reactors used a K-type thermocouple placed in the middle of the catalyst bed. To minimize temperature gradients within the reactor zone, an electrical resistance heating block was used for temperature control. Gaseous effluent was analyzed online using an Inficon micro GC (Model 3000A) equipped with MS-5A, Plot U, alumina, and OV-1 columns, and a thermal conductivity detector. Liquid samples collected from the knockout pot were analyzed separately ex situ using liquid chromatography (for aqueous products) and gas chromatography-mass spectrometry (GC-MS) (for hydrocarbon products).

Catalyst characterization—Oligomerization liquid products comprised complex mixtures of olefins. GC-MS was used to characterize the products and estimate carbon numbers. Quantification of the mass composition of particular compounds or groups of compounds was performed using two parameters: 1) the flame ionization detector (FID) GC integrated area for a particular peak, and 2) identification based on the best match to the mass spectrometry library data. Simulated distillation profiles of the olefin product were determined following the method ASTM D2887 using the GC results of an FID equipped gas chromatograph. Mass yields were based on GC-FID response.

### Example 12

In this example, a 4:1 (mol C) mixture of butene/ethene was used as model feed for oligomerization studies using solid acid catalysts. Catalytic performance as a function of solid acid catalysts (e.g., HY, MCM-41, BEA) was evaluated and results are summarized in Table 2. In this example, for all three catalytic materials shown in Table 2, the ethene conversion was approximately 50% and the conversion of n-butene is slightly higher (57-74%). Without being limited to a single operating theory, it currently is believed that the results suggest that BEA offers higher conversion of both ethene and butene; however, at the expense (in at least some examples) of a lower product selectivity to olefin (desired) versus paraffin (undesired). For example, butane in the C₄ fraction is 5.3% with BEA, while only 1.6% and 1.3% was observed for HY and MCM-41, respectively.

TABLE 2
Catalytic activity for 4:1 (mol C) mixtures of butene and ethene
in N₂ (5 vol. % butene) over acid catalysts HY, MCM-41,
and BEA. Reaction conditions: 2 g of catalyst, 0.11 mL/min butene
feed, WHSV = 2.0 h⁻¹ (g_{butene}/g_{cat}/h).

| | | | Pres- | Ethene | Butene | Ethane/ | Butane/ |
|---|---|---|---|---|---|---|---|
| | | Temp | sure | Conv. | Conv. | Ethene | Butene |
| Entry | Catalyst | (° C.) | (psig) | (%) | (%) | (mol/mol) | (mol/mol) |
| 1 | HY | 230 | 200 | 46.7 | 62.1 | 0.0006 | 0.0161 |
| 2 | MCM- | 230 | 200 | 45.2 | 57.5 | 0.0003 | 0.0127 |
| | 41 | | | | | | |
| 3 | BEA | 230 | 200 | 48.7 | 73.6 | 0.0006 | 0.0529 |

### Example 13

In this example, catalytic performance for oligomerization of a mixture of butene and ethene over BEA zeolite was evaluated as a function of olefin feed concentration and operating temperature. For comparison purpose, ethene feed (without butene) was also evaluated as a function of operating temperature and pressure. As the olefin feed concentration is decreased from 63% to 17.0% butene (keeping the same 4:1 molar carbon ratio of ethene in the feed), the ethene conversion nearly doubles from 25% to 48%; however, the butene conversion remains approximately the same at 50%. When the operating temperature was increased from 200° C. to 230° C. the ethene conversion and butane/butene product ratio were essentially unchanged. Further, the butene conversion only marginally increased from 52% to 60%.

When ethene is fed without butene, under otherwise identical conditions at 200° C., the ethene conversion decreases from 25% (with butene) to 6% (without butene). In this example, the result suggests that ethene is in fact co-oligomerizing with butene. Additionally, with only ethene in the feed, saturation of the predominantly C₄ product is pronounced (with 94% butane). By comparison, with butene in the feed the C₄ product saturation is minimal (with 2.9% butane). When the temperature of the ethene-only feed is increased from 200° C. to 230° C. the ethene conversion largely remains at a low level (<7%). However, when the pressure is increased from 200 to 500 psig the ethene conversion increases significantly from 4.9% to 91%.

In this example, however, this high of a conversion also comes with increased saturation of the product C₄ (with 89% butane). Taken together, but without being limited by a single theory of operation, it currently is believed that these results in this example suggest that ethene is co-oligomerized with butene and it can be accomplished over BEA zeolite with reasonable conversions (up to 50% ethene and butene conversions) and with minimal saturation of the olefinic products (<3% alkanes).

TABLE 3
Catalytic activity for 4:1 (mol C) mixtures of butene and ethene
in N2 over BEA zeolite. Reaction conditions: 2 g of catalyst, 0.11
mL/min butene feed, WHSV = 2.0 h⁻¹ (gbutene/gcat/h).

| | | | | Ethene | Butene | Ethane/ | Butane/ |
|---|---|---|---|---|---|---|---|
| | | Temp. | Pressure | Conv. | Conv. | Ethene | Butene |
| Entry | Feed | (° C.) | (psig) | (%) | % | mol/mol | mol/mol |
| 1 | 63.0 vol % 1- | 200 | 200 | 25.3 | 52.3 | 0.000 | 0.029 |
| | butene | | | | | | |
| | 32.5 v % | | | | | | |
| | ethene | | | | | | |
| 2 | 17.0 v % 1- | 200 | 200 | 48.4 | 45.4 | 0.000 | 0.016 |
| | butene | | | | | | |
| | 8.5 v % ethene | | | | | | |
| 3 | 63.0 v % 1- | 230 | 200 | 24.2 | 59.6 | 0.000 | 0.032 |
| | butene | | | | | | |
| | 32.5 v % | | | | | | |
| | ethene | | | | | | |
| 4 | Ethene only | 200 | 200 | 6.1 | — | 0.000 | 0.938 |
| 5 | Ethene only | 230 | 200 | 4.9 | — | 0.000 | 0.425 |
| 6 | Ethene only | 230 | 500 | 90.8 | — | 0.002 | 0.888 |

### Example 14

In this example, catalytic performance results for mixed butene/ethene oligomerization whereby Ni was incorporated into BEA zeolite, SiO₂—Al₂O₃ (referred to as “Olig-1” in Table 4, below), and MCM-41 solid acid catalysts are shown in Table 4 for select tests. The effect of different Ni loadings and operating temperatures was also investigated. When Ni loading of BEA was increased from 1% to 5% the ethene conversion increased from 40% to 69% (Table 4, entry 1 and 5). Further, the butene conversion increased from 55% to 69%. Most interestingly, this boost in conversion came with no consequence of increased olefin saturation. In both cases the alkane content of the C₄ cut was less than 2%. Additional enhancement to conversion was achieved by increasing the operating temperature from 200° C. to 230° C. Here the ethene and butene conversions were increased from 69% to 78%, and 69% to 73%, respectively (Table 4, entry 5 and 6). There was also little consequence of olefin saturation under these conditions (<2% butane in C₄ cut). While ethene and butene conversion levels and degrees of saturation were achieved in some examples with Ni incorporated SiO₂—Al₂O₃ (“Olig-1”) and MCM-41 supports, the products observed for these catalysts were not, in some embodiments, as selective to the jet-range as for Ni-promoted BEA.

TABLE 4
Catalytic activity for 4:1 (mol C) mixtures of butene and ethene in
N₂ (63 vol. % butene) over Ni-promoted BEA zeolite. Reaction conditions: 2
g of catalyst, 0.11 mL/min butene feed, WHSV = 2.0 h⁻¹ (g_{butene}/g_{cat}/h).

| | | | | Ethene | Butene | Ethane/ | Butanes/ |
|---|---|---|---|---|---|---|---|
| | | Temp | Pressure | Conv. | Conv. | Ethene | Butene |
| Entry | Catalyst | (° C.) | (psig) | (%) | (%) | mol/mol | mol/mol |
| 1 | 1 wt. % | 200 | 200 | 40.2 | 55.3 | 0.003 | 0.016 |
| | Ni/BEA | | | | | | |
| 2 | 1 wt. % | 230 | 200 | 46.3 | 59.5 | 0.012 | 0.029 |
| | Ni/BEA | | | | | | |
| 3 | Olig-1 | 200 | 200 | 41.0 | 38.6 | 0.001 | 0.031 |
| 4 | Olig-1 | 230 | 200 | 45.3 | 43.2 | 0.004 | 0.025 |
| 5 | 5 wt. % | 200 | 200 | 68.6 | 69.3 | 0.033 | 0.012 |
| | Ni/BEA | | | | | | |
| 6 | 5 wt, % | 230 | 200 | 77.6 | 73.0 | 0.097 | 0.018 |
| | Ni/BEA | | | | | | |
| 7 | 2 wt. % | 200 | 200 | 65.4 | 65.0 | 0.019 | 0.013 |
| | Ni/MCM-41 | | | | | | |
| 8 | 2 wt. %/Ni | 230 | 200 | 78.6 | 76.0 | 0.076 | 0.014 |
| | MCM-41 | | | | | | |

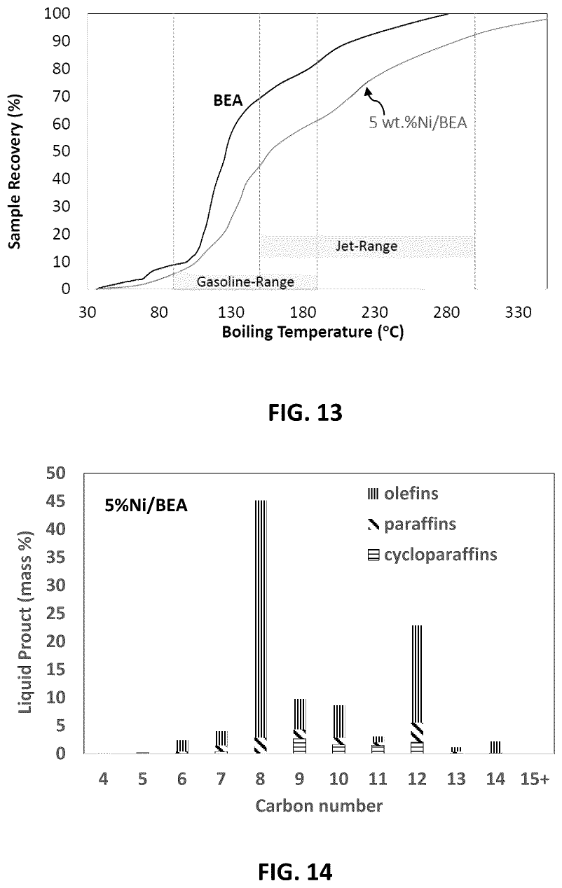

FIG. 13 shows the simulated distillation profile for the liquid products produced from BEA zeolite both with and without Ni promotion (5 wt. % Ni). Without Ni promotion, the liquid product comprises mostly olefins in the gasoline range, with only 30% in the jet-range (Table 2, entry 3). By contrast, with 5 wt. % Ni promotion, the selectivity towards the jet-range is higher with approximately 50%. In both cases, the lighter-than-jet olefins can be recycled to improve overall carbon efficiency to the jet-range.

FIG. 14 shows the GC-MS analysis for the liquid products produced from BEA zeolite with Ni promotion (5 wt. % Ni). The liquid product comprises primarily C₈ and greater carbon numbers. Further, this analysis shows the liquid product consists of 80%, 11%, and 9% olefins, paraffins, and cycloparaffins, respectively. This example suggests the lighter-than-jet olefins can be recycled over the oligomerization catalyst to produce primarily olefins in the jet-range. After oligomerization, the olefins are hydrotreated and distilled to produce a jet blend stock rich in iso-paraffins.

FIG. 14 accompanies FIG. 13 (simulated distillation) by showing the corresponding liquid product analysis for carbon number and hydrocarbon classification (olefin, paraffin, etc.). This example demonstrates the method to make a high olefin containing liquid product. This example also demonstrates that the lighter-than-jet olefins can be recycled to high jet-range, and it is possible to make predominantly olefins even in the presence of a high Ni loading content (5 wt. %). This example also shows if the olefins are saturated (to paraffins), the lighter-than-jet olefins cannot be recycled to the jet-range.

In view of the many possible embodiments to which the principles of the present disclosure may be applied, it should be recognized that the illustrated embodiments are only preferred examples of the present disclosure and should not be taken as limiting the scope of the present disclosure. Rather, the scope of the present disclosure is defined by the following claims. We therefore claim as our present disclosure all that comes within the scope and spirit of these claims.

We claim:

1. A method, comprising:
contacting, in a single reactor, a feedstock comprising a mixture of C₂ and C₃₊ olefins with a hybrid catalyst system comprising a transition-metal based catalyst and an acid catalyst; and
heating the single reactor at a temperature ranging from 250° C. to 500° C. to convert the feedstock into an oligomerized product composition comprising jet-range olefins via co-oligomerization.

2. The method of claim 1, wherein the oligomerized product composition further comprises non-jet-range products.

3. The method of claim 2, further comprising:
separating the jet-range olefins from the non-jet-range products; and/or
contacting the non-jet-range products with the hybrid catalyst system to produce jet-range olefins.

4. The method of claim 1, wherein contacting the feedstock with the hybrid catalyst system comprises (i) contacting the feedstock with the transition-metal based catalyst, which is provided in a first catalyst bed contained within the single reactor to produce a first product mixture and then contacting the first product mixture with the acid catalyst, which is provided in a second catalyst bed contained within the single reactor to convert the first product mixture into the oligomerized product composition; or (ii) contacting the feedstock with the transition-metal based catalyst and the acid catalyst, wherein the transition-metal based catalyst and the acid catalyst are provided on the same catalyst bed in the single reactor

5. The method of claim 1, wherein the oligomerized product composition comprises 0 wt % to less than 5 wt % aromatics.

6. The method of claim 1, wherein the feedstock is derived from a heterogeneous carbonaceous feed source via methanol synthesis and a methanol-to-olefin process.

7. The method of claim 1, wherein the feedstock comprises mixed olefins with a C₃₊:C₂ molar ratio ranging from 5:1 to 1:5.

8. The method of claim 1, wherein the C₃₊ olefins comprise olefins having a carbon number of C₃, C₄, C₅, or a combination of such olefins.

9. The method of claim 1, wherein the jet-range olefins comprise olefins having carbon numbers ranging from C₈ to C₁₆.

10. The method of claim 1, wherein the temperature ranges from 250° C. to 300° C.

11. The method of claim 1, wherein the method is carried out under a pressure ranging from 75 psig to 300 psig.

12. The method of claim 1, wherein the transition-metal based catalyst comprises nickel (Ni).

13. The method of claim 12, wherein the transition-metal based catalyst is Ni/SiO₂—Al₂O₃.

14. The method of claim 1, wherein the acid catalyst comprises a ZSM-5 zeolite having a Si/Al ratio ranging from 10 to 250.

15. The method of claim 1, wherein the method provides a single pass conversion of the mixture of C₂ and C₃₊ olefins into jet-range olefins of at least 90% after a time on stream of at least 60 hours.

16. The method of claim 2, wherein a ratio of the jet-range olefins to the non-jet-range products is at least 65%.

17. A reactor for performing the method of claim 1, the reactor comprising a hybrid catalyst system comprising a transition-metal based catalyst and an acid catalyst, wherein the transition-metal based catalyst and the acid catalyst are provided on separate catalyst beds or on the same catalyst bed and the hybrid catalyst system is provided in an amount sufficient to convert C₂ and C₃₊ olefins into jet-range olefins via co-oligomerization.

18. The reactor of claim 17, wherein the reactor is further connected to (i) a feedstock source comprising the mixture of C₂ and C₃₊ olefins or a first feedstock source comprising the C₂ olefins and a second feedstock source comprising the C₃₊ olefins; (ii) a high-pressure syringe pump manifold
comprising one or more syringes; or (iii) a combination of (i) and (ii).

19. A combination, comprising:
a feedstock comprising a mixture of C₂ and C₃₊ olefins; and
a hybrid catalyst system comprising a transition-metal based catalyst and an acid catalyst.

20. The combination of claim 19, wherein the transition-metal based catalyst comprises Ni/SiO₂—Al₂O₃, the acid catalyst comprises ZSM-5 zeolites having a Si/Al ratio ranging from 10 to 250.

21. A method, comprising:
contacting, in a single reactor, a feedstock comprising a mixture of C₂ and C₄ olefins with a single catalyst component comprising a transition-metal based catalyst and an acid catalyst; and
heating the single reactor at a temperature ranging from 150° C. to 300° C. to convert the feedstock into an oligomerized product composition comprising jet-range olefins, diesel-range olefins, or a combination thereof via co-oligomerization.
